(19) United States
(12) Patent Application Publication
(10) Pub. No.: US 2013/0184739 A1
(43) Pub. Date: Jul. 18, 2013

(54) CLOT ENGAGEMENT AND REMOVAL SYSTEMS

(71) Applicants: Eamon Brady, Loughrea (IE); David Vale, Clontarf (IE); Michael Gilvarry, Headford (IE); Mahmood Razavi, Irvine, CA (US)

(72) Inventors: Eamon Brady, Loughrea (IE); David Vale, Clontarf (IE); Michael Gilvarry, Headford (IE); Mahmood Razavi, Irvine, CA (US)

(21) Appl. No.: 13/643,970

(22) PCT Filed: Apr. 28, 2011
(86) PCT No.: PCT/IE2011/000026

§ 371 Date: Apr. 2, 2013

Related U.S. Application Data
(60) Provisional application No. 61/282,950, filed on Apr. 28, 2010.

(51) Int. Cl. A61F 2/01

(52) U.S. Cl. CPC A61F 2/01
USPC 606/200

(57) ABSTRACT

A device for the removal of clot obstructing the flow of blood through an arterial vessel comprises an elongate member, a clot engaging element and a capture basket. The elongate member extends in use from a point adjacent a target treatment site interior of the patient to a point exterior of the patient. The capture basket comprises a frame and a net, and has an expanded and a collapsed configuration. The clot engaging element comprises a plurality of struts having an expanded and a collapsed configuration. The plurality of struts form a first section and a second section, the first section tapering outward and distally from the elongate member and connected to the second section. The second section comprises a plurality of cells defined by a plurality of struts and arranged around at least a portion of the circumference of an axis substantially parallel to that of the elongate member. The clot engaging element and the capture basket are restrained in the collapsed configuration for delivery to the target site. The clot engaging element is located adjacent the distal end of the elongate member and proximal of the capture basket.

The invention relates to devices, and methods of removing acute blockages from blood vessels. The invention especially relates to removing acute obstructions from blood vessels. Acute obstructions may include clot, misplaced devices, migrated devices, large emboli and the like. More particularly the invention relates to removing clot from cerebral arteries in patients suffering acute ischemic stroke.

Accessing the neurovascular bed is difficult with conventional technology as the target vessels are small in diameter, are remote relative to the site of insertion and are highly tortuous. Despite the fact that there are over 600,000 acute ischemic strokes in the US each year, clot retrieval devices are used to treat patients in less than <1% of cases. The reasons for this are that conventional technology is either too large in profile, lacks the deliverability to navigate tortuous vessels or is not effective at removing clot when delivered to the target site.

There are significant challenges associated with designing clot removal devices that can deliver high levels of performance. Firstly there are a number of access challenges that make it difficult to deliver devices. In some patients the configuration of the aortic arch makes it difficult to position a guide catheter in the larger arteries that supply blood to the brain. These difficult arch configurations are classified as either type 2 or type 3 aortic arches with type 3 arches presenting the most difficulty. The tortuousity challenge is even more severe in the arteries approaching the brain. It is not unusual at the distal end of the internal carotid artery that the device will have to navigate a vessel segment with a 180° bend, a 90° bend and a 360° bend in quick succession over a few centimetres of vessel.

Secondly, neurovascular vessels are more fragile than similarly sized vessels in other parts of the body and are in a soft tissue bed. This issue is compounded by the fact that in many instances the clot is firmly wedged in the vessel. Typically a few hours have passed before the patent arrives at the hospital, is appropriately screened and arrives at the catheterisation lab for treatment. During this time a number of processes are in play that strongly bonds the clot to the vessel wall. Firstly the clot is under the influence of pulsing blood pressure and this pulsing blood pressure progressively force-fits the clot to the vessel. Some additional clot will also be laid down adjacent the occlusion. After the initial occlusion, endothelial cells between the clot and the vessel wall are compromised and bonds are formed between the vessel wall and the clot. All three of these mechanisms play a role in strongly adhering the clot to the vessel wall. Breaking these bonds without damaging these fragile vessels is a significant challenge. The high aspect ratio of the device and the vessel tortuousity make it difficult to transmit forces to the clot and for the user to feel reaction forces from the clot.

These and other problems are solved by the present invention.

### STATEMENTS OF THE INVENTION

A number of embodiments of the invention are disclosed herein. In the statements below the main embodiments are firstly described as a whole, with a list of further embodiments relating to variants of specific features or uses appended below the main embodiments. It will be appreciated that these further embodiments/feature variants may also be applicable to any of the main embodiments.

A device is disclosed for the removal of an occlusive clot from a vessel wherein the occlusion has substantially cut off blood supply to a distal vascular bed, the device comprising a basket and a clot holding assembly, the basket comprising a frame, a net and an elongate member, the frame comprising a first ring member and having a collapsed delivery configuration, a deployed configuration and an expanded configuration for dislodging clot from a vessel wall, the first ring member configured to be expanded distal of the occlusive clot, the basket further comprising a cable extending through the lumen of the elongate member, the cable attached to the first ring member, the cable comprising an activated state and a deactivated state, in the activated state the cable transmitting a force from the user to the frame, said force causing the deployed frame to assume the expanded state.

In other embodiments this invention may further include one or more of the following:

The expansion of the frame may comprise an articulation of at least a portion of the frame.

The frame may further comprise a connector element and a collar arrangement.

The expansion of the frame may comprise an articulation of the first ring member.

The frame articulation may comprise an angular displacement of the frame.

The frame articulation may comprise a change in the shape of the frame.

The expansion of the frame may comprise an articulation of the connector member.

The deployed state may comprise a partially expanded state.

The frame may be biased towards the deployed state.

The frame may return to the deployed state when the cable is deactivated.

The frame may comprise a cable attachment to which the distal end of the cable is fixed.

The frame may comprise at least one cable guide.

The cable guide may at least partially encapsulate the cable.

The cable guide may comprise a channel which restrains the cable.

The channel of the cable guide may be configured such that the cable can slide in the channel.

The channel may comprise an eyelet.

The channel may comprise a restraining feature.

The axis of a portion of the cable may run substantially parallel to the neutral axis of the first ring along at least a portion of the circumference of the first ring.

In the expanded configuration the diameter of the frame may be substantially the same as the diameter of the vessel in a region of occlusion.

In the expanded configuration the diameter of the frame may be substantially the same as a diameter of the clot.

The elongate member may comprise a tubular member.

The elongate member may extend in use from the region of the occlusion through the vasculature to a user interface external of the patient.

The elongate member may comprise a spring, or a polymer tube, or a hypo tube over at least a portion of its length.

The elongate member may comprise a plurality of wire filaments wherein said filaments are arranged so as to define an inner lumen.

The wire filaments may be wound in a spiral arrangement.

The wire filaments may be packed tightly together and define an inner lumen.

The basket and the clot holding assembly may be configured to be delivered through the lumen of a microcatheter.

The basket and the clot holding assembly may be restrained in the collapsed state during delivery through the microcatheter.

The frame may be restrained in the collapsed state during delivery by a restraining element.

The restraining element may comprise a tether, or a tube, or a core to which the frame is fixed.

The restraining element may be removed distal of the clot and the frame deployed.

The restraining element may comprise the inner wall of the microcatheter.

The first ring member may be configured in the expanded state to engage with the occlusive clot at the interface between the clot and the vessel wall.

The clot holding assembly may be configured to provide an abutment.

The basket may be moveable relative to the holding assembly.

The clot holding assembly may be configured to provide a clot engaging surface.

The basket may not be moveable relative to the holding assembly.

The holding assembly may comprise an engagement frame and an elongate tube.

The holding assembly may be configured to be expanded proximal of the basket frame.

The holding assembly may be configured to transmit a holding force from the user to the proximal face of the clot.

The holding assembly may hold the clot in a fixed position while the first ring member of the basket is retracted over the clot.

The holding assembly may hold the vessel in a fixed position while the first ring member of the basket is retracted over the clot.

The basket may be held in a fixed position while the clot holding assembly is retracted with the clot.

The basket and clot holding assembly may be retracted together with the clot.

The first ring member of the basket may apply an action force to the clot to dislodge the clot from the vessel wall.

The holding assembly may apply a reaction force to the proximal end of the clot.

The reaction force may reduce the portion of the action force that is transmitted to the vessel wall.

The holding assembly may be configured to allow the user to apply a greater action force to the clot distal end.

The holding assembly may protect the vessel from force applied to the clot by the basket.

The abutment may comprise an abutment surface.

The abutment surface may comprise a plurality of tether segments.

The abutment surface may comprise a plurality of tethers lased to the second ring element.

The abutment surface may comprise a plurality of strut elements.

The abutment surface may comprise a plurality of strut elements and a plurality of tether segments.

The abutment surface may be configured to hold the clot stationary while the first ring dislodges the clot from the vessel wall.

The abutment surface may be configured to distribute the engagement force over the proximal surface of the clot.

The engagement frame may comprise a second ring element.

The plurality of tether segments may be lased to the second ring.

At least one of the plurality of tether segments may be taut when the engagement frame is in the expanded configuration.

The abutment may be configured to distribute force across a surface of the clot.

In the expanded configuration the second ring element may comprise a hoop.

The hoop may comprise a flat hoop.

The hoop may comprise a zig-zag hoop.

The abutment surface may comprise a flat surface.

The abutment surface may comprise an undulating surface.

The abutment surface may comprise two or more interpenetrating flat surfaces.

The abutment surface may comprise a complex 3 dimensional surface.

The abutment surface may be configured to grip the clot.

The abutment surface may be configured to engage with the clot on multiple planes.

The engagement frame may comprise a wire.

The engagement frame may comprise at least a pair of wire segments.

The wire may comprise a hoop in the expanded state and the wire may comprise a pair of substantially parallel wire segments in the collapsed configuration.

The engagement frame may comprise at least a pair of struts.

In one embodiment the pair of struts may comprise a first end and a second end.

The struts may be connected to one another at the first end.

The engagement frame may be connected to the tubular member adjacent the strut first end.

The struts may be connected to each other at the second end.

In the collapsed configuration the engagement frame may comprise a pair of substantially parallel struts.

In the collapsed configuration the pair of struts may lie along the surface of the elongate member of the basket assembly.

In the expanded configuration the struts may move apart between the first and second ends to form a hoop.

The basket may comprise a connector member which connects the first ring member of the basket to the elongate member.

The frame may comprise a collar arrangement.

The collar arrangement may be configured to allow the elongate member to rotate relative to the frame.

The collar arrangement may comprise a frame collar and a proximal and distal stop.

The frame collar may be disposed over the elongate member and may be rotatable relative the elongate member.

The proximal and distal stops may be fixed to the elongate member.

Axial movement of the frame collar may be restricted by said proximal and distal stops.

The frame collar may be connected to the connector member.

The connector member may comprise an articulating member.

The net may comprise a braided, knitted or filament wound net and the net may be tubular with an open end and a closed end.

The open end of the net may be attached to the ring member.

The net may be configured to capture dislodged clot.

The net may be configured to capture clot fragments.

The net may comprise a high tensile fibre.

The net may comprise a para-aramid, meta-aramid, a UHMWPE, a polyethylene naphthalate (PEN), a stainless steel, a nitinol, a tungsten alloy or a mixture of these.

The first ring may comprise a plurality of net attachments.

The net attachments may comprise eyelets, notches, contoured surface.

The cable may comprise a plurality of filaments.

The distal end of the cable may be branched and each branch may be fixed to the frame at a separate attachment point.

In one embodiment one of the cable branches may be attached to the net.

The elongate member may comprise an inner lumen.

The inner lumen of the elongate member may comprise a smooth undulating surface.

The device may comprise a user interface and said user interface may be configured to allow the user to control the frame assembly and the holding assembly.

The user interface may comprise a handle attached to the proximal end of the elongate member.

The handle may comprise a control mechanism.

The proximal end of the cable may be fixed to the control mechanism.

The control mechanism may be configured to apply or remove tension on the cable.

The control mechanism may be activated by a thumbwheel on the handle.

In another embodiment the device comprises a device for the removal of a thrombotic or embolic occlusion of a blood vessel the device comprising: a basket, a clot engagement element, a pull cable, and a user interface,

the basket comprises a frame and a net, the frame configured to engage generally with the outer rim of the clot, the frame having a collapsed configuration and an expanded configuration,
the clot engager being disposed proximal of the basket and configured to engage with the clot,
the pull cable extending proximally from the basket to the user interface
the pull cable comprising a relaxed configuration and a tensioned configuration,
tensioning of the pull cable at least partially causing the frame to assume the expanded configuration.

In another embodiment the device comprises a device for the removal of clot obstructing the flow of blood through an arterial vessel, the device comprising an elongate member, a clot engaging element and a capture basket; the elongate member extending in use from a point adjacent the target treatment site interior of the patient to a point exterior of the patient; the capture basket comprising a frame and a net, and having an expanded and a collapsed configuration; the clot engaging element comprising a plurality of struts having an expanded and a collapsed configuration, the plurality of struts forming a first section and a second section, said first section tapering outward and distally from the elongate member and connected to the second section, said second section comprising a plurality of cells defined by a plurality of struts and arranged around at least a portion of the circumference of an axis substantially parallel to that of the elongate member; the clot engaging element and the capture basket being restrained in the collapsed configuration for delivery to the target site; and the clot engaging element being located adjacent the distal end of the elongate member and proximal of the capture basket and configured to engage with and dislodge clot from the vessel.

The capture basket frame may be self expanding.

The clot engaging element may be self expanding.

The elongate member may comprise a proximal section adjacent its proximal end and a distal section adjacent its distal end, said proximal section having a flexural stiffness greater than four times that of said distal section. The clot engaging element may comprise a central axis and a contact surface, said central axis being substantially parallel to the elongate member, said contact surface engaging with the clot and extending around at least a portion of the central axis.

The contact surface may extend around the entire circumference of the central axis.

The plurality of cells of the second section of the clot engaging element may be arranged around the entire circumference of an axis substantially parallel to that of the elongate member.

The elongate member may comprise an outer tubular element and an inner operating element.

The inner operating element may be movable relative to the outer tubular element and may extend both proximally and distally of the outer tubular element.

The clot engaging element may be attached to the distal section of the outer tubular element and the capture basket attached to the distal section of the inner operating element.

The capture net frame may be expandable to conform to the inner diameter of the vessel in which it is deployed.

The elongate member may contain an operating cable which may be connected to an element of the capture net frame and which can be advanced or retracted relative to the elongate member to control the degree of expansion of the frame.

The clot engaging element may be expandable to conform to the inner diameter of the vessel in which it is deployed.

The net may comprise a braided, knitted or filament wound net and may have an open end and a closed end.

The clot engaging element may comprise one or more tether segments which extend between some or all of the plurality of struts.

The clot engaging element may be laser cut from a tube or sheet.

The clot engaging element and the capture net may be restrained in the collapsed configuration by the inner lumen of a microcatheter during delivery.

The capture net frame may be self adjusting, and/or the expansion of the capture net frame may be adjustable by the user.

In another embodiment a device for the removal of a thrombotic or embolic occlusion of a blood vessel the device comprises a self expanding frame for use in the treatment of embolic or thrombotic disease of a blood vessel, the frame comprising a collapsed state for delivery, a partially expanded state and a fully expanded state wherein in the fully expanded state the frame comprises an unrestricted opening of substantially the same size as the cross-section of the target vessel, the frame further comprising at least one cable attachment point and at least one cable guide, wherein the cable guide restrains the cable in a path substantially parallel to the path of the strut but spaced apart from the neutral axis of the strut and allows the cable to slide relative to the cable guide.

In another embodiment a device for the dislodgement and removal of an occlusive clot in a blood vessel comprises a self expanding frame, the frame comprising a collapsed state for delivery, a partially expanded state and a fully expanded state wherein in the fully expanded state, the frame comprises a first hoop and a second hoop, the first hoop and the second hoop are connected at an articulating junction, the angle of the first and second hoops with respect to each other being variable.

The articulating junction may comprise a strut.

The first hoop, the second hoop and the articulating junction may be integral

The first hoop, the second hoop and the articulating junction may be cut from a self expanding tube.

The first hoop, the second hoop and the articulating junction may be cut from a self expanding sheet.

The first hoop, the second hoop and the articulating junction may comprise a shaped wire.

The device further may comprise an elongate member and the elongate member is connected to the frame adjacent the distal end of the elongate member.

In use the elongate member may extend from the region of the occlusion through the vasculature to the exterior of the patient.

The elongate member may comprise an inner lumen.

The device may comprise a cable, said cable fixed to an attachment point on the frame and extending through the vasculature to the exterior of the patient.

The cable may comprise an activated state wherein tension is applied to the cable by the user and a deactivated state wherein the cable is substantially free of tension.

In the cable activated state the frame may articulate.

The cable may comprise a plurality of activated states.

The frame may comprise a plurality of fully expanded states.

The frame may comprise two pairs of struts connected by the articulating junction in the collapsed state.

The frame may comprise a pair of elliptical rings in the deployed state.

The pair of elliptical rings may comprise a major axis and a minor axis.

The major axis of the pair of elliptical rings may be substantially aligned with the central axis of the vessel in the deployed state.

The cable attachment point may be adjacent the distal end of the distal ring when the ring is in the deployed state.

Activation of the cable may cause the both rings to rotate relative to the axis of the vessel.

Activation of the cable may cause the rings rotate in opposite directions.

Activation of the cable may cause the distal end of the distal ring moves towards the proximal end of the proximal ring.

The centre of the frame may comprise the crossing point of the major axis and the minor axis and the centre of the frame may be substantially coaxial with the central axis of the elongate member.

The centre of the frame may be spaced apart from the axis of the elongate member.

The frame may comprise a connector member, the connector member configured to connect the proximal hoop of the frame to the elongate member.

The net may be attached to the frame.

The net may be attached to the distal hoop of the frame.

The net may be attached to the proximal hoop of the frame.

The net may pass over the distal hoop of the frame.

The net may pass through the opening defined by the distal hoop of the frame.

The distal hoop may be slidable relative to the net.

The articulating junction may comprise an area where the thickness of the frame is reduced.

The articulating junction may comprise an area where the width of the frame is reduced.

The articulating junction may comprise a strut or a wire connecting the first and second hoops.

The articulating junction may comprise a pair of struts or wires connecting the first and second hoops.

The pair of struts or wires may be connected to each other.

The articulating junction may comprise a tether connecting the first and second hoops.

The articulating junction may comprise a weakened section.

The articulating junction may comprise a stress distributing region.

The frame may be fixed to the elongate member.

The frame may be rotatable relative to the elongate member.

The cable may extend parallel with the elongate member.

The cable may extend through the lumen of the elongate member.

Another embodiment of this device is for use in the dislodgement of an occlusive clot in a vessel the device comprising an expandable distal section and an elongate tubular member, wherein the expandable distal section comprises a collapsed configuration for delivery and an expanded configuration for dislodgement of the occlusive clot, the self expanding distal section comprising a plurality of self expanding members, said self expanding members projecting radially outward from the distal section of the elongate tube, each self expanding member comprising an atraumatic end, an engagement section and an attachment, the engagement section projecting substantially radially outwardly relative to the elongate tube and the attachment fixed to the elongate tube.

The elongate tube may comprise a lumen and said lumen may be configured to slidably receive a clot removal assembly, wherein the clot removal assembly comprises a shaft and a clot removal element and the shaft extends through the lumen of the tubular member and the clot removal element is distal of the expandable distal section of the device.

The clot removal element may comprise a basket.

The clot removal element may comprise a clot engagement device.

The expandable distal section may comprise a clot engagement device.

The atraumatic end of the at least one self expanding member may comprise a curved surface.

The atraumatic end of the at least one self expanding member may comprise an eyelet.

The atraumatic end of the at least one self expanding member may comprise a soft material.

The atraumatic end of the at least one self expanding member may comprise a curved member.

The self expanding distal section may comprise an abutment surface.

The abutment surface may comprise an annular surface.

The abutment surface may comprise a tapered surface.

The abutment surface may be concentric with the lumen of the elongate tube.

The abutment surface may be offset relative to the lumen of the elongate tube.

The abutment surface may comprise a plurality of tether segments.

At least one of the tether segments may comprise at least a partially circumferential segment.

At least one of the tether segments may comprise at least a partially radial segment.

At least two of the self expanding members may be connected.

In another embodiment a device for the dislodgement and removal of an occlusive clot in a blood vessel comprises a basket assembly, the basket assembly comprising a self expanding frame, a net and an elongate member, the frame comprises a hoop and a support ring segment, the hoop connected the elongate member and configured to appose a vessel circumference, the support ring segment is fixed to the hoop and the support segment is configured to appose a portion of a circumference of the vessel, the support segment providing an engagement support to the hoop.

The hoop may comprise a collapsed delivery configuration and an expanded configuration for engagement and dislodgement of the occlusive clot.

The hoop may be biased towards the expanded configuration.

The hoop may comprise a first strut and a second strut and each strut may be configured to form one half of the hoop.

The support ring segment may comprise a first end and a second end and the first and second ends may be attached to the first and second struts.

The centre of the hoop may be substantially coaxial with the axis of the elongate member.

The centre of the hoop may be spaced apart from the axis of the elongate member.

The elongate member of the basket assembly may be sized such that it can be interfaced with a clot debonding device.

In another embodiment a device for the dislodgement and removal of an occlusive clot in a blood vessel comprises a basket assembly, the basket assembly comprising a frame, a net, a cable and a tubular member, the frame comprising a self expanding hoop and a support, the support comprising a curved strut wherein the curve is configured to interact with the surface of the vessel, the ends of curved strut articulating with respect to the hoop and being fixedly connected to the hoop.

The curved strut may be integral with the hoop.

The frame may comprise a one piece self expanding structure cut from a tube.

The structure may comprise an articulation region connecting the curved strut to the hoop.

The articulation region may comprise a region wherein the wall of the tube is reduced.

The articulation region may comprise a region wherein the width of the section is reduced.

The curved strut may comprise a hinge at each of its ends.

The net may be attached to the hoop.

The frame may comprise a collapsed configuration, a deployed configuration and an expanded configuration.

The expanded configuration may comprise the clot dislodgement configuration.

The collapsed configuration may comprise the delivery configuration.

The deployed configuration may comprise the clot removal configuration.

In the collapsed configuration the struts that form the hoop may be compressed together to facilitate delivery.

In the collapsed configuration the curved strut may be collapsed to facilitate of the frame through a microcatheter.

In the deployed state the hoop may expand.

The cable may comprise an activated state and a deactivated state.

In the activated state the cable may be in tension and may cause the curved strut to articulate relative to the hoop.

In the activated state the cable may be in tension and may cause the expanded hoop to articulate relative to the elongate member.

In the deactivated state the cable may not be in tension and the frame may return to its deployed state.

The tubular member may comprise an abutment surface at its distal end.

The abutment surface may engage with the frame when the cable is activated.

The tubular member may be integral with the frame.

### BRIEF DESCRIPTION OF THE DRAWINGS

FIG. 1a and 1b are isometric views of a device for removing an obstruction to a vessel, FIG. 1a is the device in an expanded configuration and FIG. 1b is the device in a collapsed configuration;

FIG. 2a is an isometric view of another device of the invention;

FIGS. 2b-2e are side views of the distal end of the device of FIG. 2a;

FIG. 2f is an isometric view of the proximal end of a device of the invention;

FIG. 2g shows a portion of a frame support tube;

FIG. 2h is a cross section of a frame support tube;

FIG. 3a is an isometric view of another device of the invention;

FIGS. 3b-e are close up views of the basket frame of FIG. 3a;

FIGS. 3f-3g are isometric views of basket constructions;

FIG. 4a is an isometric view of a basket frame;

FIG. 4b is a partial cross section of a basket in a microcatheter;

FIG. 4c is an isometric view of a debonder;

FIG. 4d is an isometric view of the device;

FIG. 4e is a partial cross section view of the device inside a vessel;

FIG. 5a is an isometric view of a basket frame;

FIG. 5b is an end view of the basket frame of FIG. 5a;

FIG. 6a is an isometric view of a basket frame;

FIG. 6b is an end view of the basket frame of FIG. 6a;

FIG. 7a is an isometric view of a basket frame;

FIG. 7b is an end view of the basket frame of FIG. 7a;

FIG. 8a is an isometric view of a basket frame;

FIG. 8b is an end view of the basket frame of FIG. 8a;

FIG. 8c is an illustration of a basket frame in a collapsed configuration;

FIG. 9a is an isometric view of a basket frame;

FIG. 9b is an end view of the basket frame of FIG. 9a;

FIG. 10a is an isometric view of a basket frame

FIG. 10b is an end view of the basket frame of FIG. 10a;

FIG. 11a is an isometric view of a basket frame;

FIG. 11b is an end view of the basket frame of FIG. 11a;

FIG. 12 and FIG. 13 illustrate two basket frames of this invention;

FIGS. 14a-14g are isometric views of another device of this invention;

FIGS. 15-17 are isometric views of other devices of this invention;

FIG. 18a is an isometric view of another device of the invention in an expanded configuration;

FIG. 18b is an isometric view of the device of FIG. 18a in a collapsed configuration;

FIGS. 19a-19i illustrate a method of use for the devices described in FIGS. 15-18;

FIGS. 20a-20d are isometric views of a basket assembly;

FIG. 20e-20f are close up views of FIG. 20d;

FIG. 21a is an isometric view of another device of the invention in a collapsed configuration;

FIG. 21b is an isometric view of the device of FIG. 21a in an expanded configuration;

FIGS. 22-25 are partial cut away views of a debonding assembly;

FIG. 26a-FIG. 26c are views of another the device of the invention;

FIGS. 27-30 are isometric views of a device of the invention in various configurations;

FIG. 31a is an isometric view of a device of the invention;

FIG. 31b is an elevation view of the device of FIG. 31a in a collapsed configuration;

FIG. 31c is an elevation view of the device of FIG. 31a in an expanded configuration;

FIG. 31d-FIG. 31f illustrate the device of FIG. 31a in use;

FIG. 32a is an isometric view of another device of the invention;

FIG. 32b-FIG. 32c illustrate the device of FIG. 32a in use;

FIG. 33a is an isometric view of another device of the invention with a debonder in a delivery configuration;

FIG. 33b is the device of FIG. 33a with the debonder assembly in an expanded configuration;

FIGS. 34 and 35 are close up views of two debonders;

FIGS. 36a-36i show a device of the invention in use;

FIG. 37a is an isometric view of another device of the invention;

FIG. 37b is a plan view of the untensioned basket frame of FIG. 37a;

FIG. 37c is a side view of the untensioned basket frame of FIG. 37a;

FIG. 37d shows the basket of FIG. 37a in a collapsed configuration;

FIG. 37e shows the debonder of FIG. 37a in a collapsed configuration;

FIG. 38a is an isometric view of a debonder assembly;

FIG. 38b is and end view of the debonder of FIG. 38a;

FIG. 39a is a close up view a basket frame;

FIG. 39b is an isometric view of a basket frame;

FIG. 40 is an isometric view of a basket frame;

FIGS. 40a-40c are close up views of a basket frame of FIG. 40; and

FIGS. 41a-43b are isometric views of basket frames according to the invention.

Sheet 1 of drawings: Eccentric Basket & Debonder. Hoop type debonder (self expanding). Basket frame with tether guides. Net connection eyelets. Hoop debonder. Slotted tube type debonder.

Sheet 2 of drawings: Eccentric Basket & Debonder. Hoop type debonder with strings. Swivel hoop basket with net and activation tethers. Control handle to tension tethers

Sheet 4 of drawings: Eccentric Basket and Debonder. Hoop type debonder (self expanding). Double hoop basket with tether activation

Sheet 6 of drawings: Double Hoop Eccentric Basket with radially projecting strut type debonder. Method of use.

Sheet 8 of drawings: Basket Frames. Tether activated. Eccentric with support strut.

Sheet 11 of drawings: Tether Activated Baskets. Collapsed and expanded. Structural elements. Self expanding/hinged.

Sheet 13 of drawings: Tether Activated Baskets. Tether connected centrally to frame. Tether guided along frame and connected to distal end of hoop.

Sheet 14 of drawings: Tether Activated Baskets. With hoop type debonder.

Sheet 15 of drawings: Tether Activated Baskets. With hoop type debonder. Method of use.

Sheet 16 of drawings: Tether Activated Baskets. With hoop type debonder. Method of use.

Sheet 17 of drawings: Tether Activated Baskets. With hoop type debonder. Method of use.

Sheet 18 of drawings: Tether Activated Baskets. Flat hoop frame Frame. articulated by tether. Distal end of wire articulated by tether.

Sheet 19 of drawings: Tether Activated Baskets. Detailed construction of articulating end of frame wire.

Sheet 21 of drawings: Debonder. Hoop style debonder. Tether activated. Constructions.

Sheet 22 of drawings: Debonder. Hoop style debonder. Configuration when used with basket.

Sheet 23 of drawings: Debonder. Hoop style debonder. Configuration when used with basket.

Sheet 24 of drawings: Debonder. Hoop style debonder. Configuration when used with basket

Sheet 25 of drawings: Basket and Debonder. Slotted tube debonder. Slotted tube basket frame.

Sheet 28 of drawings: Double Hoop Basket. Eccentric basket. Eccentric clot debonder. Delivery and deployment.

Sheet 29 of drawings: Double Hoop Basket. Method of use.

Sheet 30 of drawings: Double Hoop Basket. Method of use.

### DETAILED DESCRIPTION

The present invention is related to an apparatus and methods for the removal of obstructions in vessels. The present invention is directed towards the treatment of occlusions to blood vessels, especially arterial vessels and more particularly the removal of occlusive clots from cerebral arterial vessels.

Accessing cerebral vessels involves the use of a number of commercially available products and conventional procedural steps. Access products such as guidewires, guide catheters and microcatheters are described elsewhere and are regularly used in procedures carried out in cerebral vessels. It is assumed in the descriptions below that these products and methods are employed in conjunction with the device and methods of this invention and do not need to be described in detail.

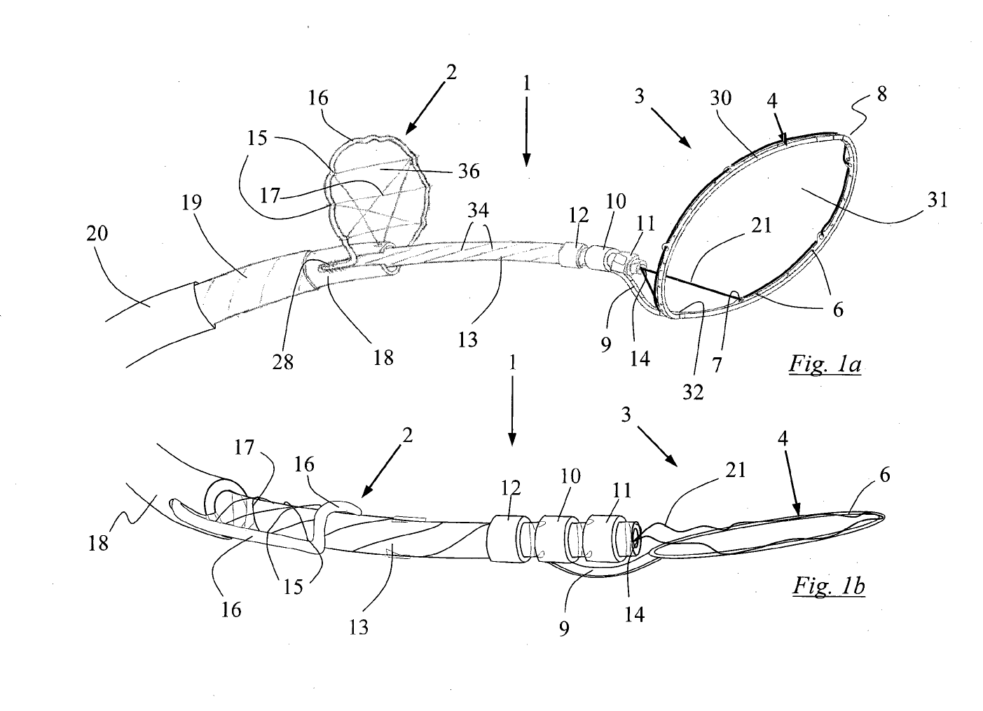

With reference to FIG. 1 there is shown a schematic representation of a device 1 for the removal of an obstruction 40 to a vessel 41. The device 1 comprises a clot debonder device 2 and a clot engagement and capture basket 3. The capture basket 3 comprises a collapsed configuration for delivery and an expanded configuration for clot engagement and capture. The clot engagement and capture basket 3 is biased towards the expanded configuration. The clot debonder 2 comprises a collapsed state for delivery through the vasculature and an expanded state for engagement with the clot and for disengaging the clot from the vessel wall.

The capture basket 3 further comprises a frame 4 and a capture net 5. The frame 4 comprises rigid strut members 30 configured to from a hoop. The frame 4 comprises a capture opening 31. The capture opening 31 comprises a hoop shaped opening or an elliptical shaped opening or a circular shaped opening. In one embodiment the frame 4 comprises a metallic frame manufactured from either a wire or a tube. In one embodiment the frame is manufactured from nitinol. The frame 4 may be manufactured from a hypo tube. This allows the struts 30 of the frame 4 to be shaped and profiled, including drilling the eyelets 6 in the frame.

In one embodiment the frame comprises eyelets 6 for the attachment of a net 5. The eyelets are preferably machined in the frame. Eyelets may be laser drilled in the frame 4. The frame 4 may be a profiled frame. The frame 4 comprises cable guides 7. The cable guides 7 comprise holes or guiding features in the frame through which a small high tension cable 21 passes. The cable guides 7 are configured such that the cable 21 (not shown) can slide through the cable guide 7.

The cable guides 7 are configured so as to guide the cable 21 substantially parallel to the axis of the strut 30 over at least a portion of its length. The cable guide 7 is further configured so as to ensure that when the cable 21 is under tension that it is spaced apart from the neutral axis of the strut 30 of the frame 4 over at least a portion of its length. In spacing the cable 21 from the neutral axis the cable 21 imparts a bending moment to the strut 30 when tensioned. This bending moment has the effect of changing the shape of the frame 4. The bending moment may be used to change the angle of articulation of the frame 4 relative to the axis of the frame support tube 13. To do this the cable 21 is axially spaced apart from the neutral axis of the frame 4, with the cable 21 positioned axially proximal of the frame 4.

In another embodiment the bending moment associated with tensioning the cable 21 is used to change the shape of the capture opening 31 of the frame 4. To do this the cable is spaced apart radially relative to the neutral axis of the frame 4. It will be appreciated that the cable 21 may be guided so as to articulate the frame over one portion of its length and to change the shape of the frame over another portion of its length.

In another embodiment two cables are employed. The first cable is used to control the articulation of the frame 4 with respect to a support element and the second cable is employed to change the shape of the capture opening 31 of the frame 4. In one embodiment the support element comprises a tubular support 13 extending proximally of the basket 3.

The basket 3 further comprises a connector member 9 that connects the hoop shaped portion of the frame 4 to the support 13. The connector member 9 may comprise a strut element and in one embodiment the connector member is integral with the hoop shaped portion of the frame 4. The connector is mounted to the support tube 13 with frame collar 10. The frame collar 10 is configured to swivel or rotate around support tube 13. This is achieved by providing proximal and distal abutment surfaces for the collar 10 to engage with. In the embodiment shown the abutment surfaces comprise first fixed collar 11 and second fixed collar 12. It will be appreciated that other abutment surface configurations are possible including: a flared on the tubular support 13, a step on the support 13, a recess on the support 13, one or more projecting tabs on the support 13 or combinations of these.

The connector member 9 is configured to lie substantially parallel to the support tube 13 in the delivery configuration and to lie at an angle to the support tube in the expanded configuration (shown). Where the angle between the connector element 9 and the support tube 13 is shallow then the axis of the support tube 13 shall be spaced apart from the centre of the capture opening 31 and the support tube 13 will be biased towards the wall of the vessel adjacent the clot 40. In one embodiment the length and angle of the connector element 9 are configured such that the axis of the support tube 13 and the centre of the capture opening 31 are substantially coaxial. In another embodiment the length and angle of the connector element 9 are configured such that the axis of the support tube 13 and the centre of the capture opening 31 are spaced apart. In another embodiment the length and angle of the connector member 9 are configured such that the axis of the support tube 13 lies between the centre of the capture opening 31 and the rim of the capture opening 31. The rim of the capture opening 31 is defined as the inner most surface of the struts 30 of the capture opening 31.

The net 5 is configured to be highly soft and flexible and is made from a yarn that is exceptionally fine. The fineness of a yarn is defined by its linear density.

The linear density, (or linear mass) is a measure of mass per unit of length, and is used to characterise yarns, strings and other similar one-dimensional objects. The SI unit of linear density is the kilogram per metre (kg/m). The linear density, μ (sometimes denoted by λ), of an object is defined as:

$$μ = \frac{\partial m}{\partial x}$$

where m is the mass, and x is a coordinate along the (one dimensional) object.

A common unit of measure of the linear density of a yarn is Dtex. Dtex is defined as the number of decigrams in one kilometer of the yarn. Thus 1 Dtex=1 dg/km=0.1 mg/m.

The net is preferably made from a Ultra High Molecular Weight Polyethylene yarn, an aramid yarn, a liquid crystal polymer yarn, an aromatic yarn, a Zylon yarn, a nitinol yarn, a stainless steel yarn, a stainless steel alloy yarn, or a tungsten yarn. It will be appreciated that these yarns may be used in conjunction with any of the baskets and debonders of this invention. The net may also be constructed from a monofilament of any of the above materials.

Commercially available UHMWPE yarns include Dyneema by DSM and Spectra BY Honeywell. The aramid yarn is preferably a para-aramid yarn. Commercially available aramid yarns include Kevlar by DuPont, Twaron, and Technora both supplied by Teijin. Commercially available LCPs include Vectra by Ticona, Vectran by Kuraray, and Zydar by Solvay Advanced Polymers. Zylon is commercially available from Toyoba.

The Table below outlines the suitable linear densities, preferred linear densities and most preferred linear density for each of the polymer yarns described above.

| Yarn | Suitable | Preferred | More preferred | Most preferred linear |
|---|---|---|---|---|
| Material | linear density | Linear density | linear density | density |
| UHMWPE | Less than 19 | Less than 10 | Less than 7 Dtex and | Less than 3 Dtex and |
| | Dtex | Dtex | greater than 1 Dtex | greater than 1 Dtex |
| Aramid | Less than 22 | Less than 12 | Less than 8 Dtex and | Less than 4 Dtex and |
| | Dtex | Dtex | greater than 1 Dtex | greater than 1 Dtex |
| Zylon | Less than 28 | Less than 15 | Less than 12 Dtex and | Less than 5 Dtex and |
| | Dtex | Dtex | greater than 1 | greater than 1 Dtex |
| LCP | Less than 25 | Less than 14 | Less than 9 Dtex and | Less than 5 Dtex and |
| | Dtex | Dtex | greater than 1 | greater than 1 Dtex |

The device shaft is an elongate member that extends in use from a point exterior of the patient to a point adjacent the target clot to be retrieved. Various shaft constructions are disclosed and described herein, including means by which the shaft may be rendered flexible for ease of delivery through tortuous vasculature. It is however also desirable that the shaft is not made so flexible that it becomes difficult to deliver a sufficient push force to advance it to the target site. To deal with this apparent conflict it is desirable that the shaft have a stiffness gradient along its length, with the flexural stiffness of the proximal region of the shaft being greater than that of the distal region. Specifically it is desirable that the flexural stiffness of the proximal region of the shaft be more than four times greater than that of the region adjacent the clot engaging portions at the distal end of the device. For the purposes of this specification flexural stiffness is defined as the stiffness measured by a 5% deflection in a three point bend test such as described by ASTM D790.

In one embodiment as shown in FIG. 1a the shaft comprises an assembly of tubular members and an inner cable. The support tube 13 extends in use from the region of the occlusion through the vasculature to the user. The support tube 13 allows the user to advance or retract the basket 3. The support tube comprises an inner lumen 33 and an exit port 14. The inner lumen 33 guides the cable 21 from the user interface 70 (not shown) which is exterior of the patient to a region adjacent the frame 4 where the cable 21 exits the lumen 33 via exit port 14. The exit port 14 is shown at the end of the tube 13. It will be appreciated that the exit port 14 may also be placed proximal of the distal end of the support tube 13. In this case the exit port 14 comprises a hole or a slot or a skive in the wall of the support tube 13. The support tube 13 is required to track through tortuous anatomy and deliver excellent mechanical force transmission to the treatment region. It is also required to facilitate relative motion between its inner lumen 33 and the pull cable 21.

It is generally recognized that providing good surface finish to metal tubes whose inner diameter is less than 0.010″ is very difficult. Normally metal tubes are formed in a cold drawing process. In order to provide for a smooth and dimensionally accurate inner lumen an inner plug is used during the drawing step. However, with very small inner diameter tubes it is not possible to support a floating plug inside the ID during drawing which results in a less accurate tolerance on the ID and a rougher surface.

In one embodiment of the invention the inner surface of the support tube 13 comprises a polished surface. The surface may be polished by injecting polishing slurry through the lumen of the support tube 13 at high pressure. In another embodiment the inner surface of the support tube 13 comprises a low friction liner. In one embodiment the liner is at least partially composed of a fluoropolymer or a polyolefin (PTFE or HDPE or URMWPE).

FIG. 1b shows another embodiment of the support tube 13 where the tube comprises a plurality of helical wires 34. With this embodiment a plurality of small diameter wires 34 are twisted to form a tube 13. The wires 34 are preferably sized greater than 30 microns and less than 80 microns. More preferably the wires 34 are between 30 microns and 60 microns. Even more preferably the wires 34 are sized between 30 and 60 micrometers. The number of wires 34 and the twist angle can be varied but which ever configuration is used the wires 34 are arranged such that a line of force contact exists between each pair of adjacent wires. This contact force keeps the wires in a tubular configuration and prevents collapse of the structure. It also ensures that the interface between the wires 34 is substantially sealed.

In another embodiment, support tube 13 comprises an inner layer of wires and an outer layer of wires 34. In one embodiment the inner layer and the outer layer have different wire diameters.

In another embodiment, support tube 13 comprises a plurality of helical wires 34 comprising non-circular cross-sections. In one embodiment the wires 34 comprise at least one substantially flat surface. In another embodiment the wires 34 comprise an elliptical cross-section.

Support tubes comprised of helical wires provide a number of important advantages that are not possible with other technologies. Firstly the inside surface of the tube is as smooth as the outer surface even at diameters of 0.010″ or less. This in combination with the undulating inner surface provides an excellent interface for relative motion between the tube and an inner member such as a core wire or a pull cable or a tether. The mechanical properties of the construction are also excellent since the twisted wire tubes have excellent push properties and good trackability features.

The construction of the elongate tube 19 may comprise a plurality of helical wires as described for the support tube 13 above. Although the inner diameter and outer diameter of the elongate tube 19 are larger than the support tube 13 the descriptions, construction details and embodiments of the elongate tube 19 are the same as for the support tube and will not be repeated.

The clot debonder 2 comprises an abutment surface 36 and an elongate tube 19. The abutment surface 36 comprises an engagement frame 16 and engagement yarn 17. The abutment surface 36 comprises a collapsed state for delivery and an expanded state for abutment with the occlusion. The distal end of the elongate tube 19 comprises a mounting section 18. The frame 16 and the elongate tube 19 are connected in the mounting section 18. In one embodiment the mounting section and the frame 16 are integral. This may be achieved by cutting the frame 16 from a metallic tube, such as nitinol, and heat treating it such that the frame is biased towards the expanded configuration. In another embodiment the mounting section 18 comprises a frame attachment 28. In one embodiment the frame attachment comprises a slot or a recess in the mounting section. The frame 16 comprises at least one projecting strut and said strut is engaged with said slot or recess. The at least one projecting strut may further be welded, glued, laminated or bonded to the mounting section 18.

The clot engagement yarn 17 of the abutment surface 36 comprises very fine yarn that is attached to the frame 16 at fixing points 15 along the frame 16. The fixing points 15 facilitate the yarn 17 being laced over and back across the opening of the frame 16 so as to create a clot engagement surface (like a tennis racquet). Preferably the yarn 17 has a slight tension in the laced configuration when expanded. This ensures that all segments of the yarn 17 engage with the proximal end of the clot 40 at the same time. It will be appreciated that the yarns will not be tensioned in the collapsed configuration. In one embodiment the fixing points 15 comprise eyelets. The frame 16 is sized such that it is the same size or smaller than the proximal lumen of the vessel. Since the primary function of the debonding element 2 is to provide an abutment surface 36 it is not necessary that the debonding frame 16 be precisely sized to the vessel. The abutment surface 36 needs to be configured to engage with at least a portion of the proximal surface of the clot 40 and provide a reaction force to the forces associated with retracting the basket 3 over the clot 40.

In another embodiment the basket 3 comprises a collapsed state for delivery, a deployed partially expanded state and an activated fully expanded state. In the collapsed state the two struts 30 of the frame 4 lie parallel to each other and substantially parallel to the axis of the micro-catheter 20 through which they are delivered. In the deployed partially expanded state the two struts move apart to form a hoop shaped frame. However the engagement force of the frame 4 is low and the hoop frame 4 makes a shallow angle with the axis of the support tube 13. When the cable 21 is activated by the user the hoop frame is articulated to a steeper angle relative to the axis of the support tube and the hoop comes into contact with the wall of the vessel. In this configuration with the cable 21 tensioned the frame 4 can engage strongly with the clot 40. The fact that the device is configured to have three configurations as opposed to two brings significant advantages. Firstly it is desired that the device strongly engage the clot such that the vessel van be recannalised rapidly without the basket pulling through the clot, a common problem with current technology. However, where the physician judges that the clot is too firmly bonded to the wall and the risks of debonding the clot are too high it is desirable that the device can be disengaged from the clot and removed. In this situation a low engagement force deployed configuration is a big advantage as otherwise removal without debonding would be problematic.

The basket of FIG. 1a can be used with the following procedural steps:
A guide catheter or sheath of between 6 F to 9 F is advanced through the vasculature until the tip of the catheter or sheath is in the carotid artery.
A guidewire and microcatheter 20 are advanced through the lumen of the guide catheter and further advanced through the internal carotid and cerebral vasculature until the tip of the microcatheter 20 is adjacent the occlusion 40.
The distal tip of the guidewire is advanced across the occlusion 40.
The microcatheter 20 is advanced over the guidewire until the tip of the microcatheter 20 is across the occlusion 40.
The guidewire is removed from the patient.
The device 1 is advanced through the lumen of the microcatheter 20 until the basket 3 emerges from the distal end of the microcatheter 20.
The basket 3 self expands to the partially expanded state.
The user activates the cable 21 at the user interface 70 and the basket 3 assumes the fully expanded state.
The microcatheter 20 is withdrawn until the tip of the microcatheter 20 is proximal of the occlusion 40.
The Debonding element 2 is advanced until the debonding frame 16 is distal of the tip of the microcatheter 20.
The debonding frame 16 self expands.
The support tube 13 is retracted by the user until the frame 4 of the basket 3 engages with the clot 40.
The debonder 2 is advanced over the support tube 13 until the debonder abutment surface 36 engages with the clot 40.
The basket 3 is retracted while holding the debonder 2 steadfast and the clot 40 is disengaged from the vessel wall.
The basket is retracted further and captures the clot.
The tether is deactivated and the frame partially collapses
The microcatheter 20, the device 1 and the clot 40 are removed from the vasculature through the lumen of the guide catheter.

In one embodiment the method involves the steps of; disengaging the basket from the occlusion, deactivating the cable, at least partially collapsing the basket, and retracting the basket across the occlusion in the partially collapsed state.

It will be noted that the use of an expansion cable allows the frame to be made with finer struts. These finer struts in general provide reduced radial force. However since the basket is relaxed in the partially expanded state there is a reduction in the strain required to collapse it fully and this feature further reduces the radial force of the frame when fully collapsed. Both of these features make it possible to deliver a high clot engagement force frame through a small microcatheter. It also makes it easier to retract the partially collapsed frame through an occlusion without causing a vessel dissection.

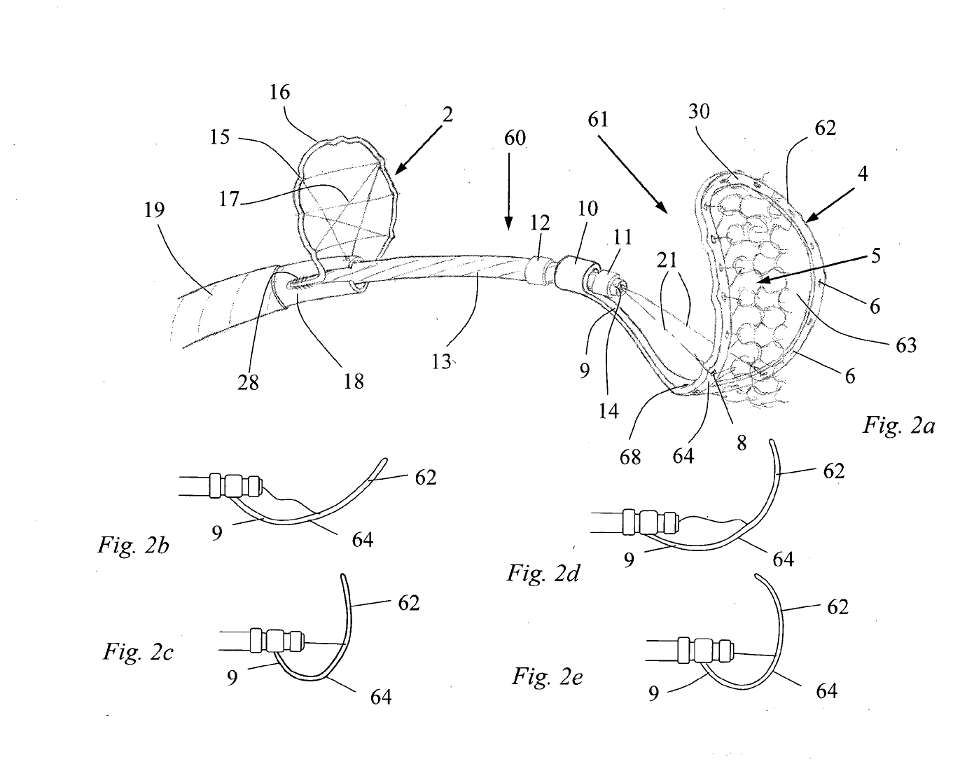

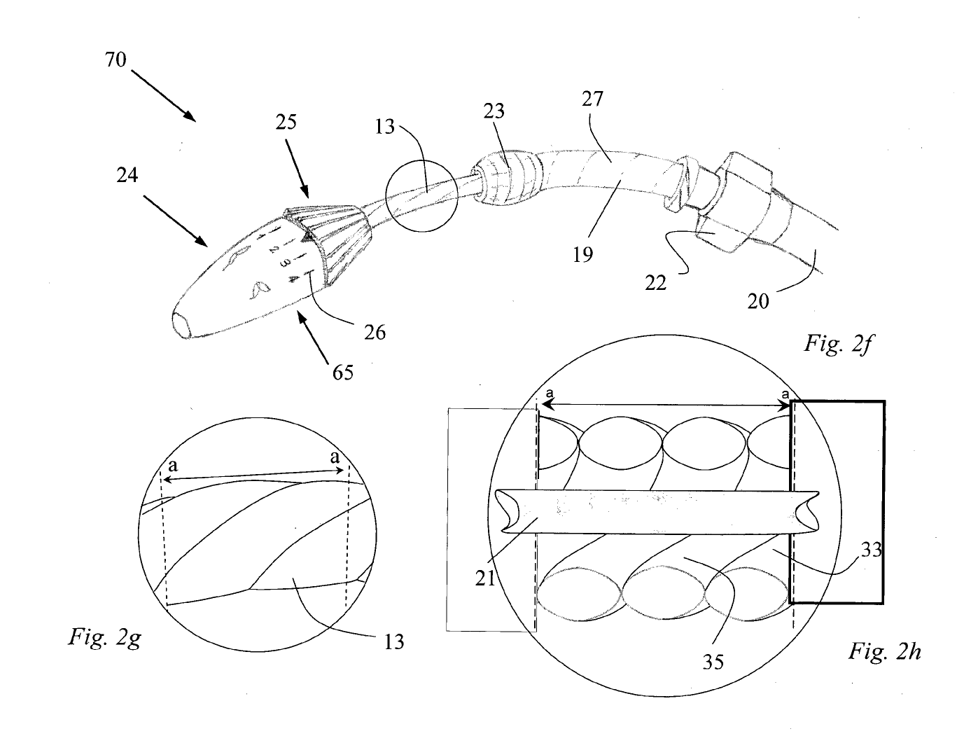

FIG. 2a shows another embodiment of the invention. The device 60 is similar to the device of FIG. 1a and FIG. 1b and similar elements carry the same numbers. The device 60 comprises a basket 61 and a clot debonder 2. The basket 61 comprises a frame 4, which is metallic and comprises a pair of struts 30 which in the expanded configuration comprise a clot capture opening 63. Unlike the frame of FIG. 1a and FIG. 1b which was a planar hoop the frame 4 of basket 61 is a curved hoop. When viewed in end-view the frame 4 of basket 61 comprises a hoop and said hoop is sized to appose the wall of the vessel in the region of the occlusion. In a side elevation view the frame comprises a C shaped element with the connector member 9 extending between the frame 4 and the support tube.

FIG. 2a shows a portion of the net 5 attached to the frame 4. The net may be a knitted net, a braided net or a weaved net. A pair of cables 21 extend from the user interface 70 through the lumen of the support tube 13 and are attached to the frame at cable attachment points 8. It will be noted that the cable attachment points 8 are fashioned in the frame 4 between the centre of the clot capture opening and the point where the connector member 9 is connected to the frame 4. With this embodiment the cables 21 pull the frame 4 towards the exit port 14 of the support tube 13. In so doing the cables 21 pull the frame 4 into a fully expanded configuration. The cables 21, when tensioned add significantly to the force with which the frame 4 engages with the occlusion. However this increase in engagement force (or engagement resistance) is directional. The frame 4 provides strong engagement when being retracted towards the clot 40 but has reduced resistance when being advanced away from the clot 40.

As with the previous design the frame 4 may be partially collapsed by deactivating the cable 21. The frame 4 of basket 61 may be cut from a flat sheet. With this embodiment the frame and connector element may be easily cut in a single pattern. With this method of manufacture no expansion steps are required and the connector is subsequently attached to the collar 10.

In another embodiment of the processing method the collar 10 is also cut from a flat sheet. With this embodiment the collar 10 is cut as a flat rectangle where the width of the rectangle is equal to the circumference of the collar. The rectangle is then rolled or formed into a collar 10. The formed or rolled collar 10 may then be welded to itself or heat set to permanently assume the shape of a collar 10.

The flat sheet frame 4 of basket 61 has a collapsed configuration, a partially expanded configuration and a fully expanded configuration. The collapsed configuration of the frame 4 is as described for FIG. 1a and FIG. 1b. The frame 4 comprises a Nitinol frame and can be heat set to a remembered shape or a biased shape. The partially expanded configuration is the biased shape of the frame 4 of basket 61. The partially expanded state requires some compressive deformation in order to fully collapse the frame and some expansive deformation in order to fully expand the frame.

FIG. 2b to FIG. 2e shows a schematic side view of the frame 4 of basket 61 in the partially expanded state and in the fully expanded state. The frame comprises a distal segment 62 and a proximal segment 64. The cable attachment point 8 separates the distal and proximal segments. When the cable 21 is activated the proximal frame segment 64 and the connector 9 change shape. The distal segment 62 of the frame 4 does not undergo significant shape change when the cable is activated. However this segment is displaced by the movement of the proximal segment 64.

FIG. 2d shows the frame 4 in the partially expanded state. The frame has a gentle C shaped curve when viewed in side elevation. If viewed in plan view the capture opening 31 would be generally elliptical or oval in shape. In FIG. 2e the frame 4 of FIG. 2b is shown in the fully expanded state (with the cable activated). The distal segment 62 of the frame 4 still comprises a gentle C shaped curve in side elevation while the proximal segment 64 and the connector 9 have a smaller radius of curvature. FIG. 2c shows another partially expanded frame 4 where the distal segment has a smaller radius of curvature than the proximal segment. In the fully expanded configuration as shown in FIG. 2e the radius of curvature of the proximal segment is now similar to that of the distal segment due to the activation of the cable 21.

In another embodiment the proximal segment 64 of the frame 4 and/or the connector 9 comprise areas of articulation. These areas are configured to bend more readily than neighboring segments and these articulation areas facilitate the shape change in the proximal segment 64 and connector 9 when the cable 21 is tensioned.

In one embodiment the cable attachment point is adjacent the neutral axis of the strut 30 of the frame 4. In another embodiment the cable attachment point is spaced apart from the neutral axis of the strut. With this embodiment the tension in the pull cable 21 sets up a torque in the strut 30 to which the attachment point 8 is fixed and this assists in changing the shape of the frame 4.

The frame 4 of FIGS. 2a to 2e is attached to the support tube 13 as described in FIG. 1a to FIG. 1b and the features of the debonder are also the same as described in FIG. 1a to FIG. 1b.

The user interface 70 is shown in FIG. 2f. The user interface comprises a handle 24 for control of the basket and a control element 23 to control the position and orientation of the debonder 2. The user interface 70 of device 60 could be applied to any of the devices of this invention which employ a cable activated basket and a debonder. The handle 24 comprises a thumb wheel 25 and a hand grip 26. The handle further comprises graduations 65 to guide the user in expanding the basket. The handle 24 is fixed to the proximal end of the support tube 13. The cable 21 extends from the proximal end of the support tube 13 into the handle 24 where it is mounted to a tension mechanism 66. The tension mechanism 66 comprises a cable wheel 67 or drum onto which the cable can be wound when being tensioned. The cable wheel 67 is rotated by activating the thumb wheel 25. The cable 21 can thus be tensioned or relaxed by rotation of the thumb wheel 25 in either a clockwise direction or an anticlockwise direction. It will be appreciated that the cable wheel 67 could also be activated with a sliding mechanism.

The control element 23 is fixed to the proximal end 27 of the elongate tube 19 and is configured to allow the user to advance or retract the debonder 2 relative to the basket 61. The control element is also configured to allow rotation of the elongate element 19. It will be appreciated that the elongate tube 19 is moveable and rotatable relative to the support tube 13.

The control element 23 is comprises a locking element such that the control element 23 and the elongate member 19 can be to the support tube 13. This allows the basket 61 and the clot debonder 2 to be fixed together and can thus advanced together or retracted together or rotated together. In one embodiment the locking element comprises a touhy-borst arrangement. In another embodiment the control element comprises a clamp. In either case the control element can be locked to the support tube 13 by the user and can subsequently be unlocked from the support tube 13 by the user.

In another embodiment the control element comprises a luer fitting such that the annular space between the support tube 13 and the elongate tube 19 can be flushed by a physiological fluid like saline. The construction of the support tube 13 and the elongate tube 19 are as described in FIGS. 252f-h.

The proximal end of the microcatheter 20 is shown in FIG. 2f with the elongate tube extending through the lumen of the microcatheter. A guide catheter is not shown but it will be appreciated that the microcatheter is inserted through the lumen of the guide catheter and the proximal end of the microcatheter 20 extends out of the proximal hub of the guide catheter (or sheath). The microcatheter hub 22 is shown and allows flushing with standard syringes and luer connectors.

FIG. 2g shows a close-up view of the support tube 13, and FIG. 2h shows a longitudinal cross-section of the same tube 13 in which the undulating inner surface 35 can be observed. The combination of the helix angle and the curved cross-section of the wire 13 ensures that the inside surface of the lumen is a smooth undulating surface 35. The undulating surface 35 provides for an excellent frictional interface with pull cables 21. In a curved vessel the pull cable 21 slides over the high points of the undulating surface 35. The cable therefore has a reduced amount of contact with the surface over its length. Furthermore, because the wires 34 are drawn before being fashioned into a tube they have a smooth outer surface which also improves the frictional interface. A portion of the outer surface of the wires 34 makes up the inner surface of the tube 13. The undulating surface reduces the frictional drag and this allows for better control of the articulation of the frame 4 with the cable 21.

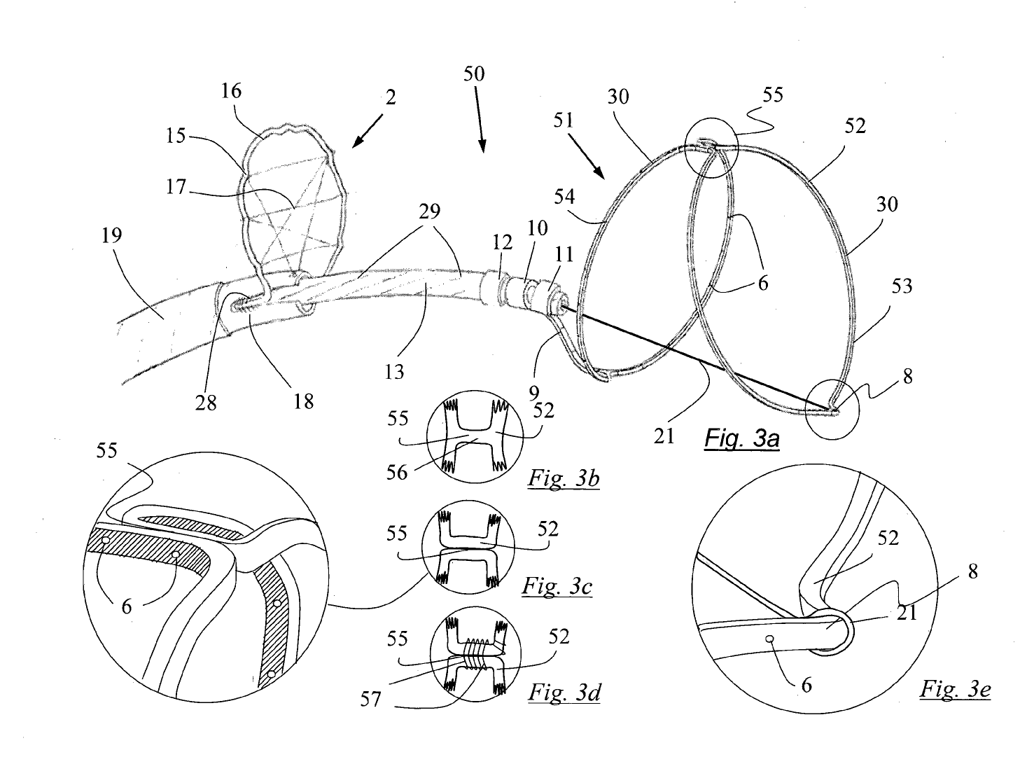

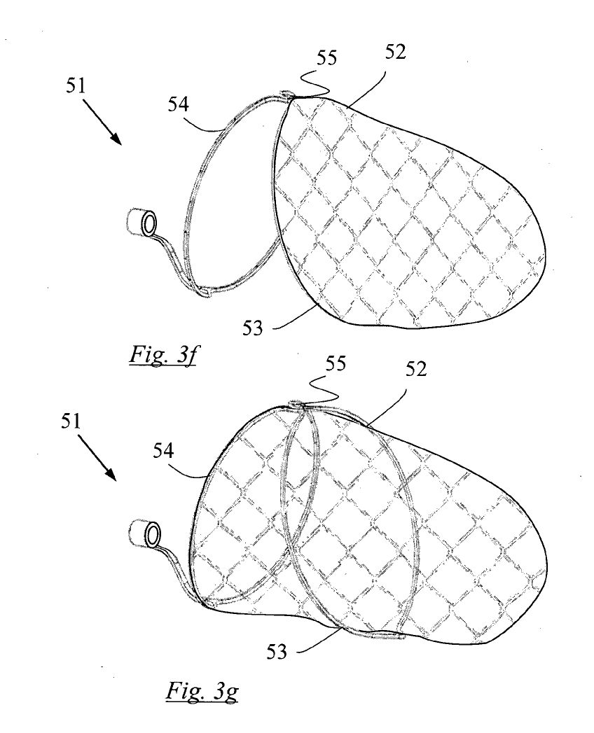

FIG. 3a shows another device 50 of the invention. The device 50 is similar to devices of FIG. 1a-b and FIG. 2a-h in that the Debonder and user interface 70 are the same. In this embodiment the basket 51 comprises a double hoop frame 52. The double hoop frame comprises a distal hoop 53, a proximal hoop 54, an articulating bridge 55 and a connector member 9. The distal hoop 53 and the proximal hoop 54 are constructed from struts 30 wherein each hoop comprises at least one pair of struts 30. The distal hoop and the proximal hoop 54 are connected to each other by articulating bridge 55. The connector member 9 is connected to the support tube 13 as described in FIG. 1a-b and FIG. 2a-h. The cable 21 is attached to an attachment point on the distal hoop 53. The attachment point 8 is radially opposite the point where the articulating bridge 55 is connected to the distal hoop 53. The cable 21 extends from the attachment point through the opening of the proximal hoop 54 and through the lumen 33 of the support tube 13 to the cable wheel 57 of the handle 24. The construction of the attachment point is shown more clearly in FIG. 3e. The cable 21 is attached at attachment point 8. The attachment point 8 may comprise an eyelet as shown in FIG. 3e. The area adjacent the attachment point 8 is an area that undergoes significant strain in moving to the collapsed configuration. FIG. 3e shows a strain relieving feature adjacent attachment point 8 which is designed to allow the struts 30 of the hoop frame to collapse to a configuration whereby they are substantially parallel while distributing the strains associated with said collapse.

In another embodiment the proximal hoop 54 comprises a cable guide 7 (as described previously) and said cable guide directs the path of the cable 21 between the attachment point 8 and the exit port 14 of the support tube 13. In one embodiment the cable guide 7 is positioned at the base of the proximal hoop 54 diametrically opposite the bridge 55. In yet another embodiment the cable guide 7 is associated with the connector.

The double hoop frame 52 comprises a collapsed delivery configuration, a deployed partially expanded configuration and a fully expanded configuration. In the collapsed state the single strut of the connector member 9 the pair of struts of the proximal hoop 54 and the pair of struts of the distal hoop 53 are connected in series and all lie substantially parallel to the axis of the microcatheter within which they are housed. When deployed from the microcatheter 20 the pair of struts 30 of the proximal hoop 54 move apart in the centre to form an elliptical or hoop shape. Likewise the struts of the distal hoop move apart to form an ellipse of hoop shaped frame. The connector member 9 expands such that it forms an angle with the axis of the support tube.

In the fully expanded configuration the cable 21 is tensioned and this draws the cable attachment point 8 proximally which causes the articulating bridge 55 to articulate and the frame 52 moves to the expanded configuration (as shown in FIG. 3a).

The bridge 55 connects the proximal hoop 54 and the distal hoop 53 and allows them to articulate with respect to one another. A number of bridge configurations are possible and some variants are shown in FIG. 3b to FIG. 3d. In FIG. 3b the bridge 55 comprises a connector strut 56. The connector strut is sized so as to be a point of flexure as the proximal and distal hoops are articulated relative to each other. The wall thickness of the connector strut may be thinner than that of the struts 30 over at least a portion of its length. The cross sectional area of the connector may be less than the cross-sectional area of a strut 30 of the frame 52. FIG. 3c shows another variant where by the bridge 55 comprises a pair of strut connectors. With this embodiment the proximal frame hoop and the distal frame hoop are not fully closed as a slight gap exists in the region of the bridge. The two open hoops are connected by two connector struts 56. The two connector struts are free to move relative to each other. FIG. 3d shows another variant which is almost identical to that described in FIG. 3c except that the two connector struts are tethered together. The tether 57 reduces the movement between the two connectors 56 and this ensures that the hoop shape is always preserved irrespective of the forces of clot engagement and capture.

FIGS. 3f and 3g show two alternative net attachment configurations. In FIG. 3f the net is attached to distal hoop 53, while in FIG. 3g the net is attached to proximal hoop 54.

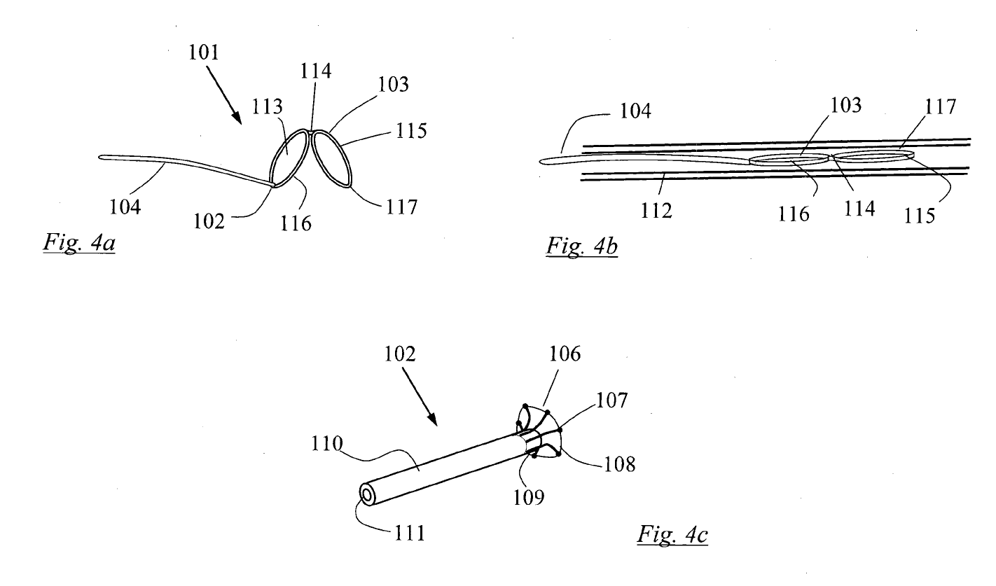

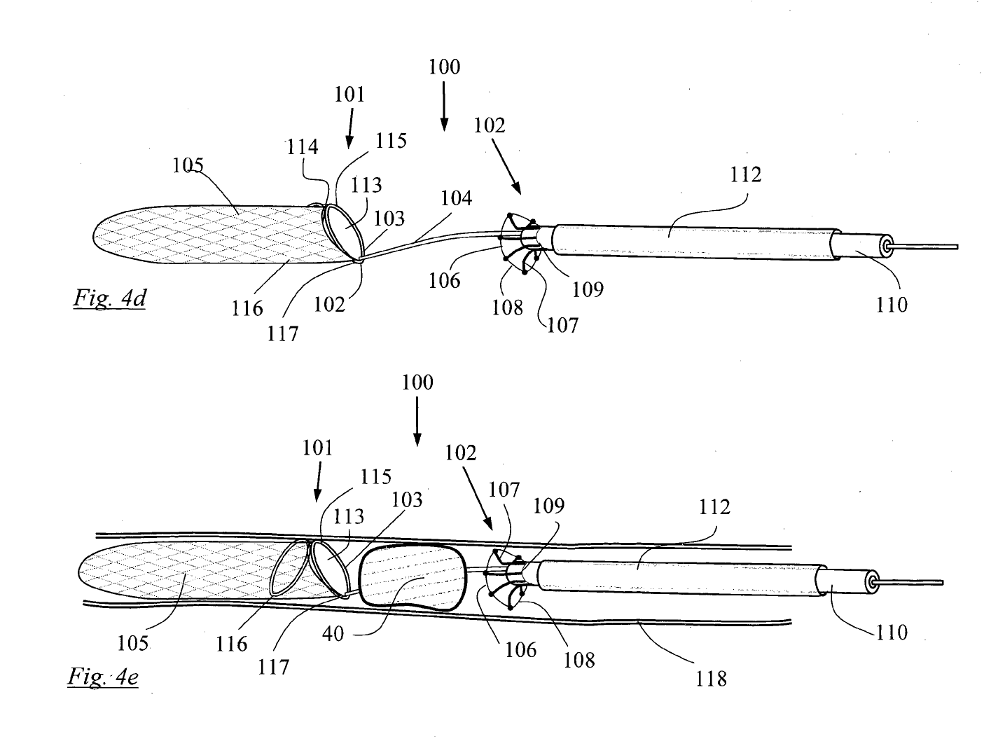

FIG. 4a through to FIG. 4e show another device 100 of the invention. The device 100 comprises a basket 101 and a clot debonder 102. The basket 101 comprises a frame 103 with a large unobstructed capture opening 113 and a support member 104 connected directly to the rim of the capture opening 113. The debonder 102 is slidable over the support member 104 and comprises a clot engagement surface 106. The clot engagement surface 106 comprises radially projecting elements 107 and circumferential filaments 108. The circumferential filaments 108 are configured to distribute the clot engagement forces between the radially projecting elements. In one embodiment the radially projecting elements 107 comprise struts. In one embodiment the radially projecting struts 107 comprise metal struts. Preferably the metal struts 107 are made from spring steel, a shape memory metal or a super elastic metal such as nitinol. The circumferential filaments 108 may comprise a thin strut, a wire, a fiber, a yarn or a multi-filament yarn. In the embodiment shown in FIG. 4c the filament 108 interconnects the radially projection struts at the outer diameter. It will be appreciated that the circumferential filaments 108 may interconnect the radially projecting struts 107 at multiple diameters. In so doing an engagement surface 106 that is comprised of a plurality of spaced apart radial elements and a plurality of spaced apart circumferential filaments. The net result is an engagement surface 106 with a spiders web pattern.

With reference to FIG. 4a the basket 101 of the clot removal device 100 is shown. The basket 101 is shown with the net 105 removed. The basket 101 comprises a double hoop frame 103 which is similar to the frame 52 of FIG. 3. However in this case the frame 103 does not employ a cable to effect deployment. The frame 103 has an enlarged deployment configuration for engagement with and removal of occlusions and a collapsed configuration for delivery through the vasculature and the frame is biased towards the deployed configuration.

The frame 103 is shown in the collapsed configuration in FIG. 4b. In this case the frame 103 is collapsed inside the lumen of a micro catheter 112. The two struts 117 of the proximal hoop 116 lie substantially parallel to one another in the collapsed state. Likewise the two struts 117 of the distal hoop 115 lie substantially parallel to one another in the collapsed state.

The frame 103 further comprises a bridge section 114 which interconnects the two hoops of the frame 103. The bridge section 114 is configured to articulate as the frame 103 moves between the collapsed configuration and the expanded configuration. In this case the bridge 114 needs to be sufficiently strong to expand the frame 103 and thus the bridge is preferably a strut or a plurality of struts.

FIG. 4c shows the debonder 102 separated from the basket 101. The debonder 102 comprises a collapsed configuration for delivery through a microcatheter 112 and an expanded configuration for engagement with an occlusion 40. The debonder is shown in the expanded configuration with the struts 107 projecting radially outward over a portion of their length. The proximal portion of the struts 107 lie substantially parallel to the axis of the debonder tube 110. In the collapsed configuration the radially projecting section of the struts 107 are collapsed such that they lie substantially parallel to the axis of the debonder tube 110. In this collapsed state the debonder 102 may be advanced through the lumen of a low profile microcatheter.

FIG. 4d shows the debonder mounted on the support member with both the basket 101 and the engagement surface 106 of the debonder 102 in the expanded configuration. The device 100 is shown projecting from the lumen of a microcatheter 112. This configuration corresponds to the device configuration just prior to the step of debonding the clot from the vessel wall. FIG. 4e shows the device 100 in the same configuration but this time in a vessel with the basket 101 and debonder 102 deployed either side of clot 40.

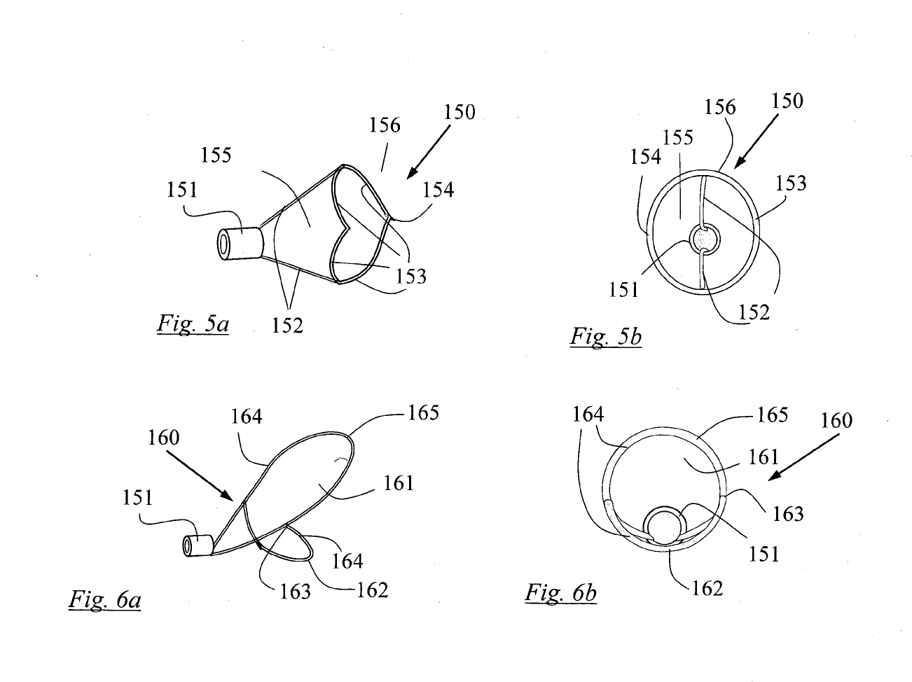

A number of different frame designs are shown in FIGS. 5a to 14g. It will be appreciated that these frames could be employed with any of the baskets in this invention.

FIG. 5a shows an isometric view and FIG. 5b shows an end view of frame 150. Frame 150 comprises a clot engagement ring 156, a pair of connector elements and a collar 151. The clot engagement ring 156 is shown in the expanded state and comprises four struts 153. The four struts 153 are arranged to form a hoop 156 in the expanded state. The hoop is configured to engage with an occlusive clot and to disengage the clot from the wall of the vessel. The clot engagement ring 156 further comprises articulation points 154 at the intersections of the four struts 153. These articulation points 154 allow the frame 150 to move between the collapsed and expanded states. The connector elements 152 ensure the frame is stable in the expanded state.

FIG. 6a shows an isometric view and FIG. 6b shows an end view of frame 160. Frame 160 comprises a hoop section 165 an articulating support 162 and a collar 151. The hoop section 165 of the frame 160 comprises struts 164 and the hoop section 165 is sized such that it is in contact with substantially the entire circumference of the vessel when deployed. The hoop section 165 of the frame 160 is sized so it will engage the clot at or adjacent to the interface between the clot and the vessel wall. In so doing the hoop section 165 of the frame 160 will be effective in peeling or delaminating the clot from the vessel wall. The hoop section 165 of the frame 160 comprises a large unobstructed capture opening 161 which allows disengaged obstruction to enter the basket without resistance. The articulating support 162 is integral with the hoop section 165 of frame 160 and provides support to the hoop section 165 of frame 160. In the expanded state the articulating support 162 engages with the wall of the vessel and prevents the collapse of the hoop section 165 as it engages with the obstruction. The frame 160 further comprises a collar 151. The collar 151 facilitates the mounting of the frame 160 on an elongate member such as a support member 104 or a support tube 13 of a basket assembly.

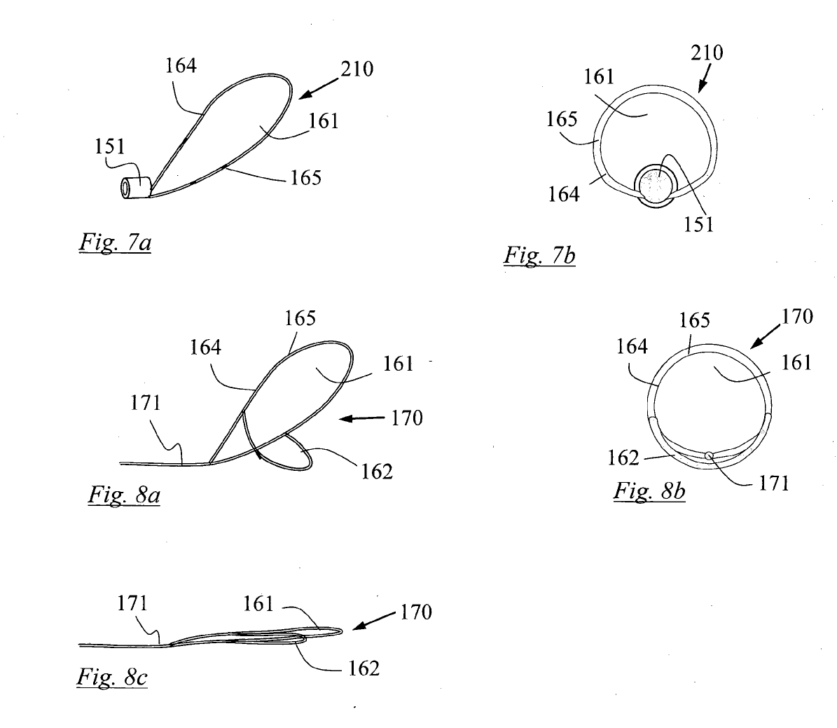

FIG. 7a shows an isometric view and FIG. 7b shows an end view of frame 210. This frame 210 is similar to the frame 160 in FIG. 6, except that it does not have an articulating support. The frame comprises a hoop section 165 and a collar 151.

FIGS. 8a and 8b shows another frame 170 which is similar to the frame 160 of FIG. 6. In this case the frame comprises a hoop section 165 an articulating support 162 and a support wire 171. The hoop section 165 and the articulating support are as described in FIG. 6. The support wire 171 may be integral with the hoop section 165 of the frame 170. In this case the support wire 171 is configured in the same way as support members or support tubes described with other baskets of the invention. In another embodiment the support wire 171 is fixed to a support member at its proximal end. FIG. 8c shows the frame 170 in the collapsed configuration with the struts of both the hoop section 165 and the articulating support 162 being substantially parallel.

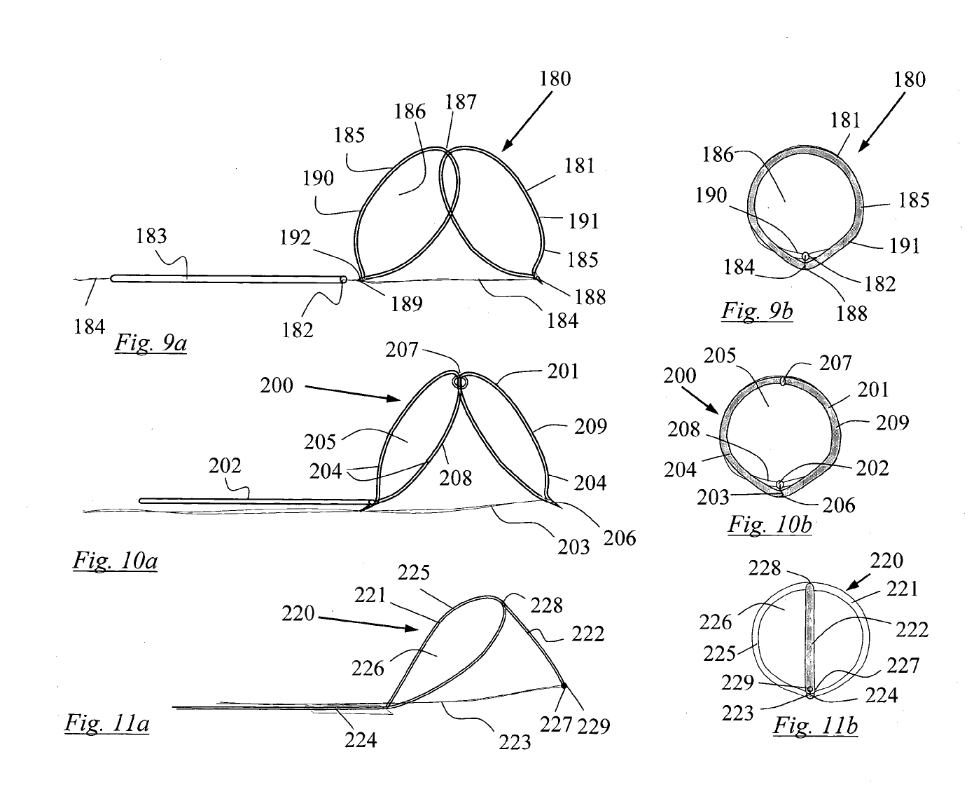

FIG. 9a shows an isometric view and FIG. 9b an end view of frame assembly 180 which is composed of a frame 181, an expansion cable 184 and a bumper tube 183. The frame 181 comprises a proximal hoop 190 and a distal hoop 191 and a bridge element 187 connecting the two hoops. The frame further comprises a cable attachment 188 on the distal hoop 191 and a cable guide 189 on the proximal hoop. The cable guide 189 comprises an abutment surface 192 on its proximal side. In one embodiment the frame 181 has a collapsed state for delivery and an expanded state for clot engagement and the frame 181 is biased towards the expanded configuration. With this embodiment the cable 184 serves to purpose of reinforcing the frame 181 in the expanded configuration such that it provides strong resistance to collapse and thus good clot engagement. With this embodiment the bridge 187 comprises an articulating element. While the bridge 187 is configured to allow the proximal hoop 190 and distal hoop 191 to articulate through a large angle of displacement it is a relatively stiff element so as to provide good radial strength to the frame 181 in the expanded state.

In another embodiment the bridge 187 comprises a flexible hinge. With this embodiment the frame has three configurations. In the collapsed delivery configuration the frame sits within the microcatheter. The two struts 185 of the proximal hoop 190 lie substantially parallel to one another in the collapsed state. Likewise the two struts 185 of the distal hoop 191 lie substantially parallel to one another in the collapsed state. In the deployed configuration the proximal hoop 190 and the distal hoop 191 expand into a hoop shape but do not articulate. The deployed frame assumes a planar configuration along the axis of the vessel. When the cable 184 is activated the abutment surface 192 of the cable guide 189 engages with the distal abutment 182 of the bumper tube 183. Further activation of the tether causes the cable attachment point 188 to move towards the cable guide 189 and this is facilitated by the hinge 187 connecting the proximal hoop 190 to the distal hoop 191. By controlling the displacement of the cable the size of the expanded frame can be adjusted by the user. The frame can be expanded such that it is in interference with the walls of the vessel, or it can be expanded such that it is closely sized to the lumen of the vessel or it can be undersized relative to the vessel. This one size fits all feature is a significant advantage of this embodiment.

In the final device configuration the frame assembly has a net attached to the frame and a debonder mounted over the bumper tube 183. The net may be attached to either the proximal hoop 190 or the distal hoop 191. Where the net is attached to the proximal hoop 190 the net must pass the struts of the distal hoop 191. In one embodiment the net passes over the distal hoop 191 and is attached to the proximal hoop 190. With this embodiment the collapsing and expanding of the frame requires the distal hoop to slide inside the net.

In another embodiment the net passes through the opening of the distal hoop 191 and is attached to the proximal hoop 190. With this embodiment the net slides through the mouth of the distal frame 191 during deployment or collapse of the frame 181.

It will be appreciated that any of the clot debonders disclosed in this invention could be employed in conjunction with the frames or frame assemblies described in FIG. 5 to FIG. 14.

FIG. 10a shows an isometric view and FIG. 10b an end view of another frame assembly 200 which is similar to the frame assembly 180 in FIGS. 9a and 9b. In this case the frame assembly 200 is composed of a frame 201, an expansion cable 203 and a support member 202. The frame 201 comprises a proximal hoop 208 and a distal hoop 209 and a bridge element 207 connecting the two hoops. The frame 201 further comprises a cable attachment 206 on the distal hoop 209. The cable 203 extends from the cable attachment 206 parallel to the support member 202 back to the user interface (not shown). The support member 202 comprises an elongate member and is fixed to the frame 201 at its distal end. In one embodiment the frame 201 has a collapsed state for delivery and an expanded state for clot engagement and the frame 201 is biased towards the expanded configuration. With this embodiment the cable 203 serves to purpose of reinforcing the frame 201 in the expanded configuration such that it provides strong resistance to collapse and thus good clot engagement. With this embodiment the bridge 207 comprises an articulating element. While the bridge 207 is configured to allow the proximal hoop 208 and distal hoop 209 to articulate through a large angle of displacement it is a relatively stiff element so as to provide good radial strength to the frame 201 in the expanded state.

In another embodiment the bridge 207 comprises a flexible hinge. In one embodiment the flexible hinge comprise a tether. With the flexible hinge embodiment the frame has three configurations as were described in FIG. 9 above.

FIG. 11a shows an isometric view and FIG. 11b an end view of another frame assembly 220. In this case the frame assembly comprises a hoop frame 221, an expansion strut 222, a cable 223 and a support member 224. The hoop frame 221 comprises a pair of struts 225 and a capture opening 226. The expansion strut is connected to the frame 221 at one end 228 and comprises a vessel wall engagement section 227 at the other end. The vessel wall engagement 227 engages the vessel wall and provides support to the frame 221 in the expanded state. In order to avoid trauma to the vessel the vessel wall engagement 227 comprises an engagement surface. Preferably the engagement surface is soft. Preferably the engagement surface engages the vessel wall over a segment of the vessel wall. The expansion strut 222 further comprises a cable attachment 229 where expansion cable 223 is attached. Expansion cable 223 and support member 224 extend proximally to the user interface (which is described elsewhere in this specification).

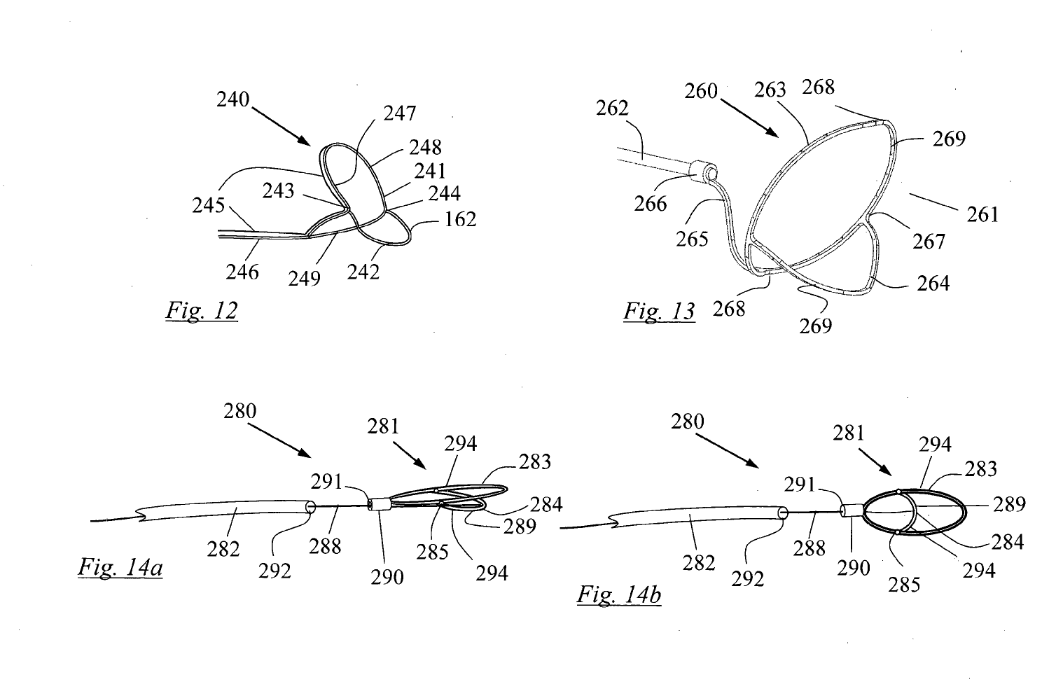

Another frame assembly 240 is shown in FIG. 12 which could be incorporated into any of the baskets of the devices of the invention. The frame assembly 240 is similar to the frame assembly described in FIG. 1 and FIG. 2 and it will be appreciated that the frame assembly 240 could be incorporated with the clot debonder 2 and user interface 70 of FIG. 1 and FIG. 2. The frame assembly 240 comprises an elastic, a shape memory or a super elastic frame and has a remembered at least partially expanded state and a deformed state. In the deformed state the frame assembly is strained by an external restraining element. In one embodiment the deformed state comprises a collapsed delivery state. In one embodiment the restraining element comprises a microcatheter. In another embodiment the restraining element comprises a tether which ties the frame assembly 240 in the restrained delivery configuration. The frame assembly 240 comprises a frame 241, a vessel engagement strut 242, a support member 246 and a pull tether 245. The frame 241 comprises a capture ring 247. The capture ring 247 comprises a vessel opposing ring in its expanded state. Said ring 247 comprises a first ring segment 248 and a second ring segment 249. Said first ring segment 248 and said second ring 249 are connected by two articulation regions 243. Said first ring segment 248 is articulated relative to said second ring segment 249 by activation of the pull tether 245.

In one embodiment the frame assembly 240 comprises cable guides 7 as described in FIG. 1. In one embodiment the pull tether 245 runs substantially parallel to capture ring 247 over at least a portion of its length. The pull tether 245 is spaced apart from the neutral axis of the capture ring 247 of the frame 241 over at least a portion of its length. The spacing of the pull tether from the neutral axis the capture ring 247 imparts a bending moment to the capture ring 247 when the pull tether 245 is tensioned. This bending moment causes the first ring segment 248 to articulate relative to the second ring segment 249 about articulation region 243. In another embodiment the pull tether 245 causes the capture ring 247 to articulate relative to the support member 246. The vessel engagement strut 242 comprises a curved, segment and the ends of said segment are connected to the capture ring 247. Preferably the vessel engagement strut 242 is integral with the capture ring 247. The vessel engagement strut 242 engages with the vessel wall and prevents the capture ring 247 from collapsing under the forces of clot engagement. The support member 246 is connected to the frame 241 and extends proximally to the user interface 70. The pull tether 245 extends proximally from the frame to the user interface 70 from where it is activated or deactivated as previously described. In one embodiment the pull tether 245 extends parallel to the support member 246 over a substantial portion of its length. In another embodiment the support member comprises a tubular member over at least a portion of its length. In one embodiment the tubular segment of the support member comprises exit port and the tether extends through the lumen of the tubular segment and exits the tubular segment via said exit port.

FIG. 13 shows another frame assembly 260 which is similar to the frame assembly of FIG. 1. However in this case the frame assembly comprises a frame 261 and a support member 262. The frame 261 comprises a one piece frame which comprises a capture ring 263, a support strut 264, a connector strut 265 and a collar 266. The frame 261 is preferably cut from a tube. In one embodiment the frame 261 is laser cut from a hypo tube. The engagement of the support strut 264 with the wall of the vessel prevents the collapse of the frame when the clot engagement ring 263 engages with the occlusion 40. The support strut 264 is connected to the engagement ring 263 at connection points 267. The connection points 267 comprise regions of bending when the frame 261 is collapsed for delivery. The engagement ring must collapse in concert with the support strut 264 and the strains of collapse are absorbed in the bending regions 268. The frame 261 further comprises net attachment points 269 on both the ring 263 and the support strut 264. In one embodiment the attachments 269 comprise eyelets in the struts of the frame 261. The connector strut 265 is configured to connect the engagement ring 263 to the support member 262. The connector strut 265 may flex such that the axis of the support member 262 may move apart from or towards the axis of the vessel while the ring 263 is engaged with the wall of the vessel. The collar 266 is fixed to the support member 262 and holds the frame 261 steadfast relative to the support member 262.

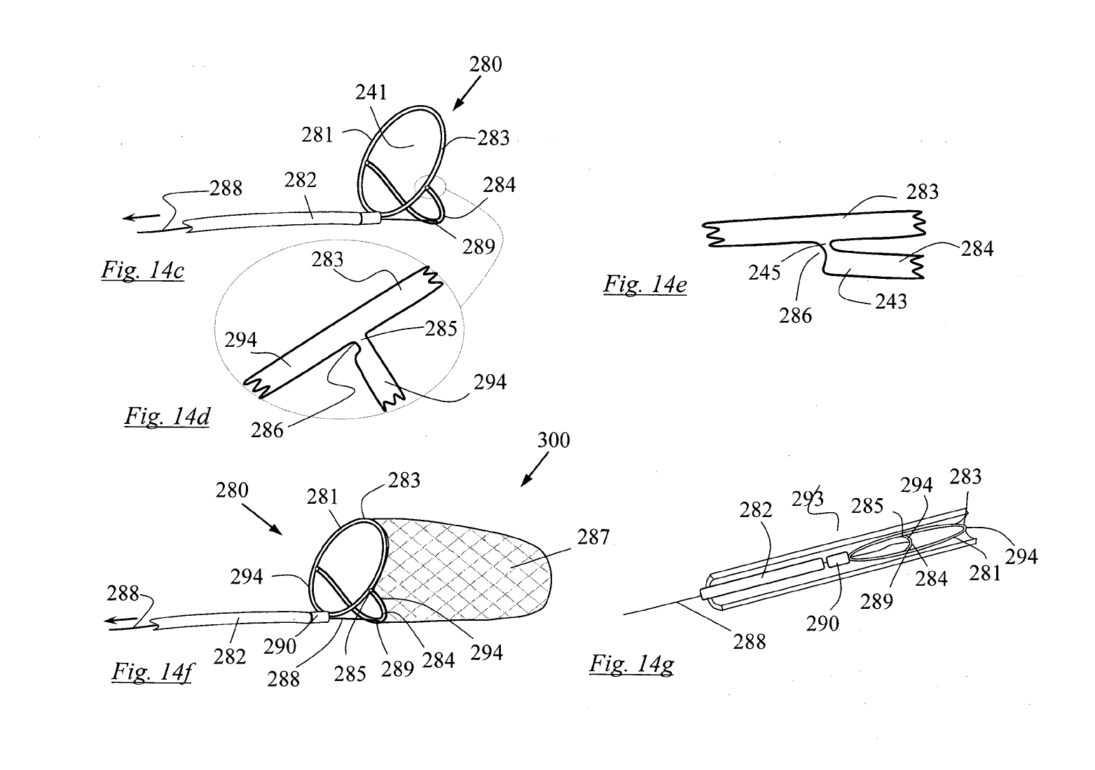

With reference to FIG. 14a to FIG. 14f there is shown basket assembly 300 including frame assembly 280. The basket assembly 300 has a collapsed state for delivery as shown in FIG. 14g, a partially expanded unconstrained configuration as shown in FIG. 14a and FIG. 14b and a fully expanded state as shown in FIG. 14c and FIG. 14f. The basket assembly 300 comprises a frame assembly 280 and a net 287. The basket assembly may be integrated with any of the clot debonder assemblies and user interfaces of the invention to create a device for the recannalization of vascular occlusions especially acute stroke occlusions.

The frame assembly comprises a frame 281, a control tube 282, and a pull cable 288. The frame 281 comprises an engagement ring 283, and a hinged support 284. The hinged support 284 is connected to the engagement ring 283 with hinges 285. The pull cable is attached to the hinged support 284 at an attachment junction 289. The attachment junction lies substantially midway between the hinges 285. The pull cable 288 extends from the attachment junction through cable guide 290 and further extends through the lumen of control tube 282. Preferably the cable 288 is activated with the assistance of a control mechanism at the user interface 70.

With reference to FIG. 14a and FIG. 14b, the frame assembly 280 is shown in the partially expanded state. The frame assembly 280 assumes the partially expanded state when the frame assembly is deployed from the microcatheter 293 with the pull cable 288 is deactivated. The pull cable 288 is deactivated when the distal abutment surface 292 of the control tube 282 is not engaged with the cable guide abutment 291. In this configuration the engagement ring 283 is expanded as is the hinged support 284 with the hinged support 284 being substantially parallel to engagement ring 283. In one embodiment the engagement ring 283 and the support 284 lie substantially parallel to the axis of the vessel in the partially expanded state. In another embodiment the engagement ring 283 and the support 284 lie substantially parallel to the axis of the control tube 282 in the partially expanded state. FIG. 14c shows the frame assembly 280 in the expanded configuration. In this configuration the pull cable 288 is activated and this causes the abutment 292 of the control tube 282 to engage with the cable guide abutment 292 of the cable guide 290. Activation of the pull cable 288 further causes the hinged support 284 to articulate relative to the engagement ring 283. The articulation of the support 284 causes the ring to articulate relative to the control tube 282 and the user may control the degree of articulation by the displacement of the pull cable 288. In this way the user may size the mouth of the engagement ring to the size of the occlusion that is to be disengaged and captured.

In one embodiment the hinge 285 comprises a pin and eyelet arrangement. In another embodiment the hinge 285 comprises a tether. In another embodiment the hinge 285 comprises an integral hinge. One embodiment of an integral hinge is shown in FIG. 14d and FIG. 14e where the hinge 285 comprises a relief section 286. The relief section 286 comprises a thinned section of the wall of the support 284. The relief section 286 allows the support 284 to articulate relative to the engagement ring 283. FIG. 14d shows a close up view of the hinge 285 when the frame 281 is in the expanded configuration. FIG. 14e shows a close up of the hinge 285 when the frame 281 is in the collapsed or the partially deployed state.

In one embodiment the integral hinge 285 is made from an elastic, a super elastic or a shape memory material and said hinge comprises a biased configuration. In one embodiment the biased configuration comprises the collapsed state. In another embodiment the biased configuration comprises the expanded state.

FIG. 14f shows a basket assembly incorporating the frame assembly 280 as described above. The basket assembly 300 is shown with the frame 281 in the fully expanded configuration. In the expanded configuration the hinged support 284 is subtended at an angle relative to the engagement ring 283.

FIG. 14g shows the frame assembly 280 in the fully collapsed configuration inside microcatheter 293. In this configuration the struts 294 of the engagement ring 283 and the hinged support 284 all lie substantially parallel to the axis of the microcatheter 293.

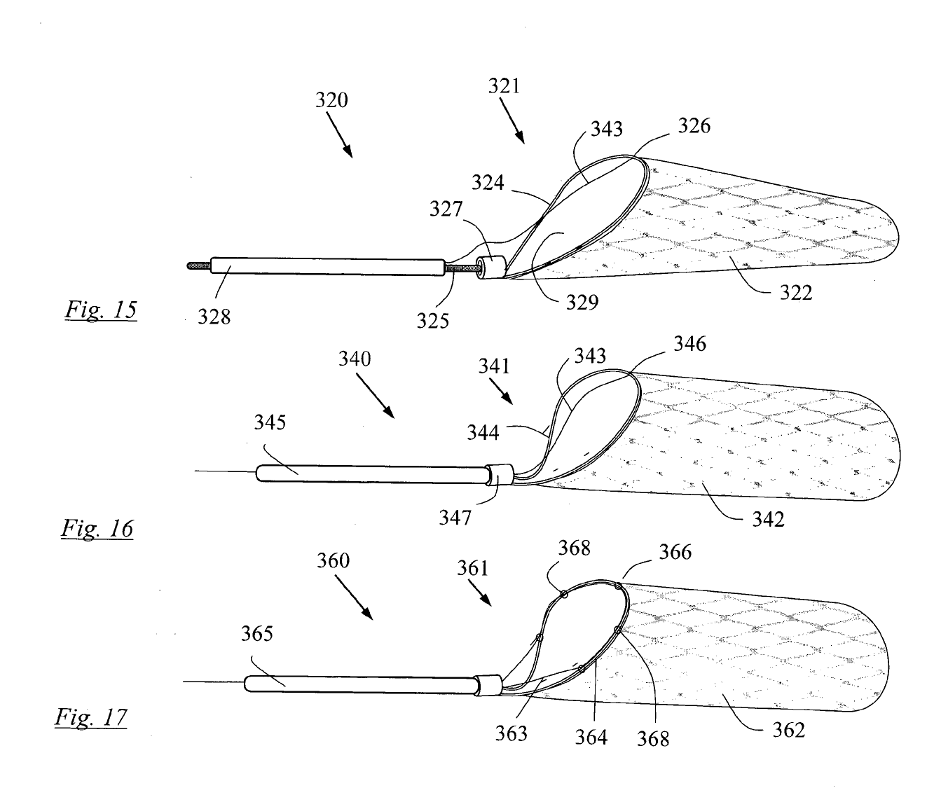

FIG. 15 shows another basket assembly 320 according to the invention. The basket assembly comprises a frame 321, a net 322, an expansion cable 323, a support member 325 and a expansion member 328. The frame comprises an engagement hoop 324 a collar 327 connecting said hoop 324 with elongate support member 325. The frame 321 further comprises cable attachment 326 which facilitates fixing of the cable 323 to the frame 321. The net 322 comprises a closed end net with openings of 500 micrometers or less configured to prevent captured clot from fragment during removal. The net 322 is attached to the frame 324 at a plurality of connection points around the circumference of the frame 321. The frame comprises a collapsed configuration for delivery, a first expanded state and a second expanded state. In the first expanded state the frame 321 self expands such that the hoop frame 324 comprises a substantially elliptical opening and said hoop frame 324 is at least partially engaged with the vessel wall. In the second expanded state the expansion member 328 is moved proximally relative to the support member 325 and this brings the cable 323 into a state of tension. The cable 323 provides additional support to the frame 321. In one embodiment the resistance of the frame to collapse is greater in the second expanded state than the first expanded state. In another embodiment the radial force of the frame is greater in the second expanded state when compared to the first expanded state.

The expansion member 328 is slidable relative to the support member 325. In one embodiment the expansion member is configured such that a debondong assembly can be mounted on its outside diameter.

FIG. 16 shows another basket assembly 340 according to the invention. The basket assembly comprises a frame 341, a net 342, an expansion cable 343, and a support member 345. The frame 341 comprises an engagement hoop 344 a collar 347 connecting said hoop 324 with elongate support member 345. The support member 345 comprises an elongate tube and the pull cable 343 extends from the attachment point 346 through the lumen of said tube to the user interface 70. The collar 347 is fixed to the support member 345. In one embodiment the support member 345 comprises a tube and the collar 347 is integral with said tube. The frame 341 further comprises cable attachment 346 which facilitates fixing of the cable 343 to the frame 341. The net 342 comprises a closed end net with openings of 500 micrometers or less configured to prevent captured clot from fragment during removal. The net 342 is attached to the frame 344 at a plurality of connection points around the circumference of the frame 341. The frame comprises a collapsed configuration for delivery, a first expanded state and a second expanded state. In the first expanded state the frame 341 self expands such that the hoop frame 344 comprises a substantially elliptical opening and said hoop frame 344 is at least partially engaged with the vessel wall. In the second expanded state the cable 343 is pulled proximally relative to the support member 345 and this brings the cable 343 into a state of tension. The cable 343 provides additional support to the frame 341. In one embodiment the resistance of the frame to collapse is greater in the second expanded state than the first expanded state. In another embodiment the radial force of the frame 341 is greater in the second expanded state when compared to the first expanded state.

FIG. 17 shows another basket assembly 360 according to the invention. The basket assembly 360 comprises a frame 361, a net 362, an expansion cable 363, and a support member 365. The frame 361 comprises an engagement hoop 364 a collar 367 connecting said hoop 364 with elongate support member 365. The support member 365 comprises an elongate tube and the pull cable 363 extends from the attachment point 346 through the lumen of said tube to the user interface 70. The collar 367 is fixed to the support member 365. In one embodiment the support member 365 comprises a tube and the collar 367 is integral with said tube. The frame 361 further comprises cable attachment 366 which facilitates fixing of the cable 363 to the frame 361. The frame 361 further comprises a plurality of cable guides 368. The cable guides 368 comprise guide elements through which the cables 363 can slide. The cable guides are configured such that the path of the cable is parallel to the neutral axis of the hoop frame 364 over at least a portion of the length of the hoop frame 364. The support member 365 comprises a lumen and the expansion cable 363 extends through the lumen of the support member 365 to the user interface 70 (not shown).

The net 362 comprises a closed end net with openings of 500 micrometers or less configured to prevent captured clot from fragment during removal. The net 362 is attached to the frame 364 at a plurality of connection points around the circumference of the frame 361. The frame 361 comprises a collapsed configuration for delivery, a first expanded state and a second expanded state. In the first expanded state the frame 361 self expands such that the hoop frame 364 comprises a substantially elliptical opening and said hoop frame 364 is at least partially engaged with the vessel wall. In the second expanded state the cable 363 is pulled proximally relative to the support member 365 and this brings the cable 363 into a state of tension. The cable 363 provides additional support to the frame 361. In one embodiment the resistance of the frame to collapse is greater in the second expanded state than the first expanded state. In another embodiment the radial force of the frame 361 is greater in the second expanded state when compared to the first expanded state.

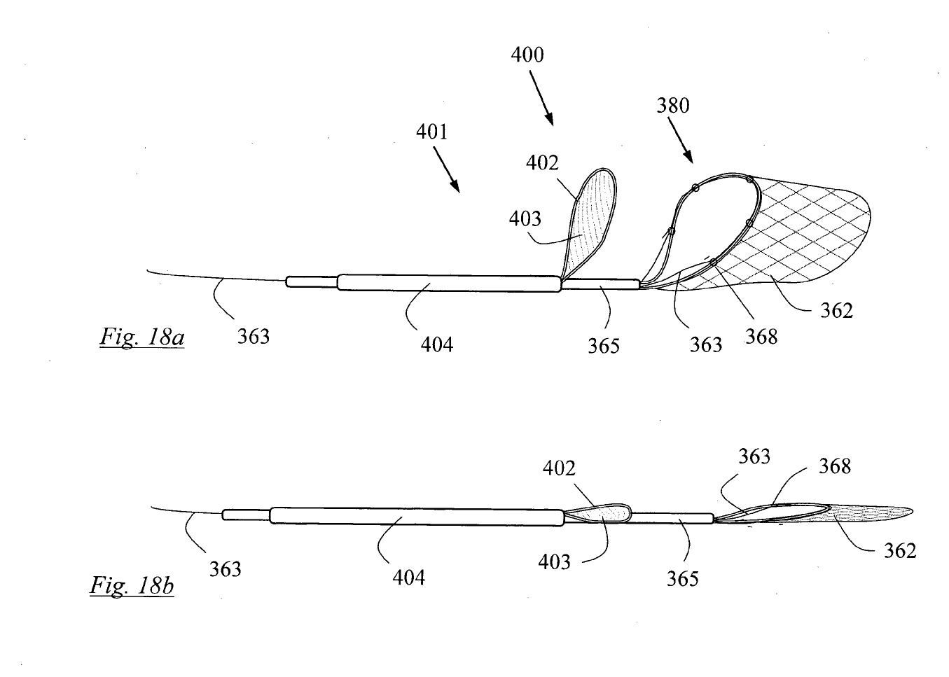

FIGS. 18a and 18b show a device 400 comprised of a basket assembly 380 and a debonder assembly 401. The basket assembly 380 is very similar to the basket assembly 360 of FIG. 17 with the exceotion that the support member is directly connected to the hoop frame. This eliminates the collar element 367 shown in the previous drawing. The basket assembly 380 functions in exactly the same way as that of basket assembly 360 of FIG. 17. The debonder 401 comprises a ring member 402 and an outer member 404. The ring member comprises a collapsed delivery configuration as shown in FIG. 18b and an expanded engagement configuration as shown in FIG. 18a. The outer member 404 comprises an elongate tube and is sized to facilitate relative movement with the support member 365 of the basket assembly 380. In the deployed configuration the ring member 402 engages with the proximal end of the clot and the ring member 402 comprises an abutment against which the basket is retracted so as to disengage the clot from the wall of the vessel without applying significant tensile forces to the wall of the vessel.

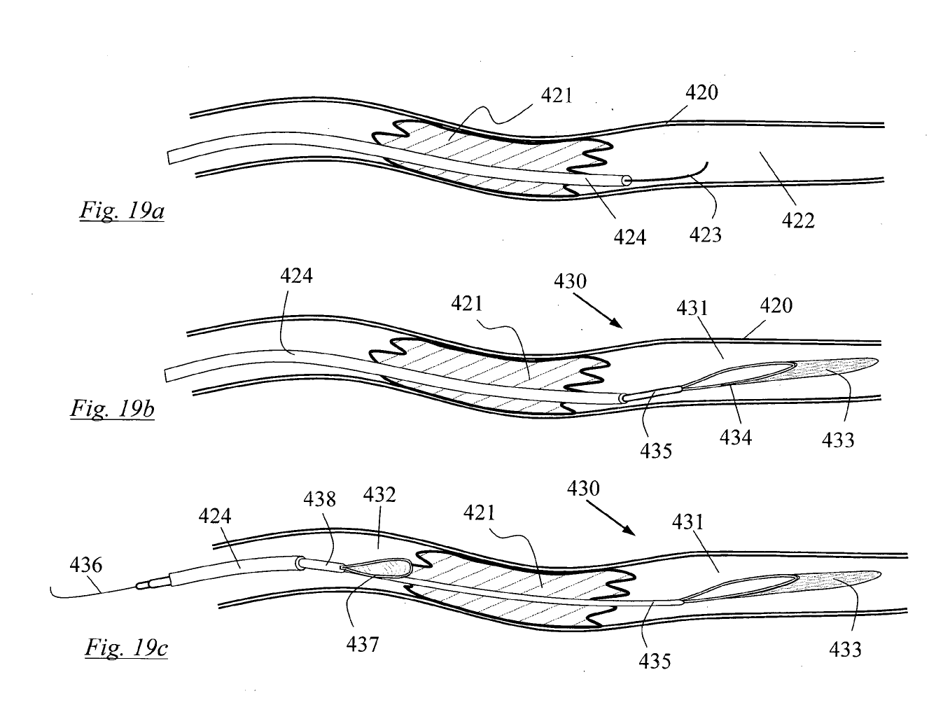

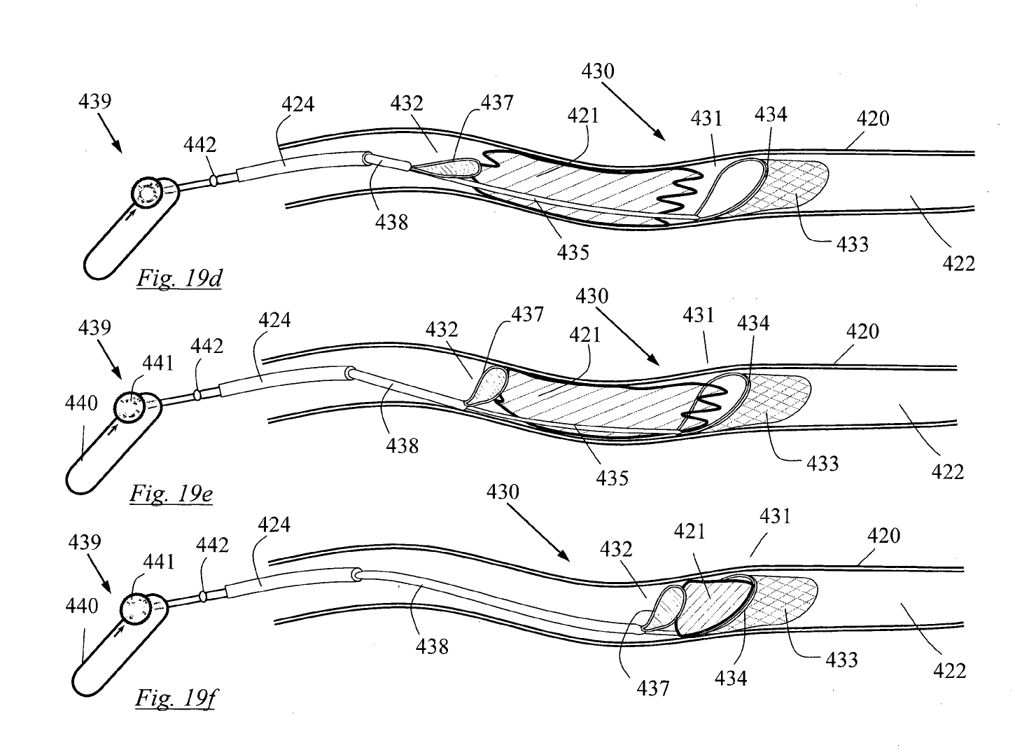

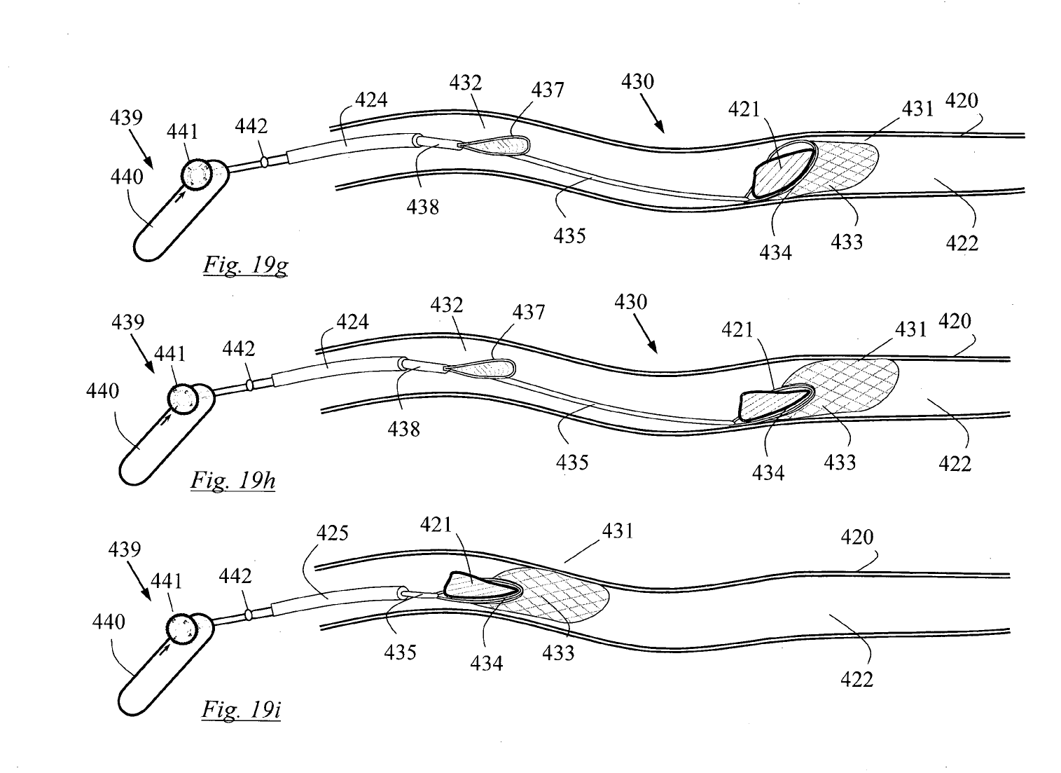

The method use of the devices described in FIG. 15 through to FIG. 18b will be described below with reference to FIG. 19a-i. FIG. 19a shows an occlusive clot 421 in a vessel 420. The occlusive clot 421 is fixed strongly to the vessel wall 422 of the vessel 420. A guide catheter is advanced into an upstream vessel of larger diameter. In the case of an occlusion of an anterior vessel of the cerebral circulation the guide catheter 425 is placed in a carotid artery. The guide catheter 425 is preferably 8 F in diameter or less. More preferably the guide catheter 425 is 7 F in diameter or less. Even more preferably the guide catheter 425 is 6 F in diameter or less. In one embodiment the procedure comprises the step of advancing a transition catheter 426 through the lumen of the guide catheter 425 such that the tip of the transition catheter 426 extends from the distal tip of the guide catheter 425 and the transition catheter 426 is advanced to into a smaller bore vessel than is possible with the guide catheter. In the example above the transition catheter 426 may be advanced into the internal carotid vessel. The tip of the transition catheter 426 may be placed in the cervical section of the internal carotid artery. For the purposes of the remaining descriptions associated with FIG. 19a-i reference to the guide catheter 425 may be interpreted to include the transition catheter 426 or not since the procedure may be conducted with or without the transition catheter 426.

With the guide catheter 425 in place (not shown) a microcatheter 424 is advanced through the lumen of the guide catheter 425 until its distal end is advanced distal of the tip of the microcatheter. The microcatheter is advanced further with the assistance of a guidewire 423 within the lumen of the microcatheter 424 and both instruments are manipulated until the tip of the microcatheter is across the occlusive clot. At this point the guidewire 423 is withdrawn.

Referring now to FIG. 19b the device 430 is advance through the lumen of the microcatheter 424 with the basket 431 and the debonder 432 in the collapsed configuration until the basket 431 is advanced distal of the tip of the microcatheter 424 with the debonder still restrained in the collapsed configuration. The basket 431 is partially expanded.

Referring to FIG. 19c, the microcatheter 424 is withdrawn while holding the basket 431 stationary until the tip of the microcatheter 424 is proximal of the occlusion 421. The debonder 432 is deployed proximal of the occlusion 421. In one embodiment the debonder 432 is deployed by withdrawing the microcatheter proximally while holding the debonder hub 442 stationary. In another embodiment the debonder 432 is deployed by holding the microcatheter 424 stationary while advancing the debonder assembly 432.

Referring to FIG. 19d, the basket 431 is fully expanded by activating the pull cable 436 with the activator 441 of the handle 440. In one embodiment the activator 441 comprises a thumbscrew. In another the cable activator 441 comprises a slider. In yet another embodiment the activator comprises a lever. The cable 436 is tensioned by the user activating the activator 441. The tension may be transmitted to the cable via a number of mechanisms. In one embodiment the mechanism comprises a rack and pinion mechanism. In another embodiment the mechanism comprises a circular drum onto which the cable 436 is wound. The fully expanded basket is withdrawn proximally until the frame 434 engages with the distal end of the clot. In one embodiment the frame comprises radiopaque elements to facilitate visualisation under fluoroscopy. The step of engaging the basket with the distal end of the clot involves sizing the vessel 420 in the region distal of the clot 421, moving the activator 441 so as to expand the frame 434, and monitoring the expansion of the frame 434 on fluoroscopy. The debonder 432 is advanced distally until the ring member contacts the proximal end of the clot 421. The debonder is advanced further to ensure the ring member 437 is fully engaged with the clot. In one embodiment the ring member 437 is deployed in a partially expanded state with the ring member deployed at an angle relative to the support member 435. With this embodiment when the ring member 437 is further advanced, engagement with the clot causes it to expand to a fully deployed configuration. Preferably the relative angle is greater than 40°.

FIG. 19e shows the basket 431 and debonder 432 fully engaged with the clot. The basket is withdrawn proximally while holding the debonder steadfast and this action breaks the bonds between the vessel wall and the clot. The debonder forces the clot into the open mouth of the basket as the basket is withdrawn. In another embodiment, as shown in FIG. 19f, the step of debonding the clot 421 from the vessel wall 420 and the step of forcing the clot 421 into the open mouth of the basket 431 comprise advancing the debonder 432 while holding the basket 431 steadfast. FIG. 19f shows the clot 421 almost completely enveloped by the net 433. The debonder 432 is advanced until the clot 421 is completely enveloped by the basket 431. In one embodiment the debonder 432 is advanced until the outer member 438 of the debonder 432 abuts the frame 434. In another embodiment the debonder 432 is configured such that at least a portion of the debonder 432 may enter the mouth of the basket 431.

In one embodiment the basket 431 and clot 421 are removed with the ring member 437 of the debonder 432 occluding the mouth of the basket 431. With this embodiment the removal steps comprise:
Slightly disengaging the debonder 432 from the proximal end of the clot 421 so as to reduce applied pressure.
Deactivating the frame 434 and partially collapsing the basket 431 by deactivating the pull cable 436.
Locking the debonder 432 to the support member 435 (thus, locking the debonder 432 to the basket 431).
Retracting the device 430 and withdrawing the basket 431 and clot 421 through the lumen of the guide catheter 425.

Referring to FIG. 19g to FIG. 19i another embodiment of the steps of removing the basket and clot comprises:
Disengaging the ring member 437 from the proximal end of the clot 421.
Retracting the debonder from the vessel segment.
Deactivating the activator 441 so as to remove the tension in the cable 436 and at least partially collapsing the basket 431.
Retracting the basket 431 proximally and removing the basket from the vessel segment.
Removing the basket 431 through the lumen of the guide catheter 425.

In one embodiment the microcatheter 424, debonder 432 and basket 431 are removed through the lumen of the guide catheter 425 together.

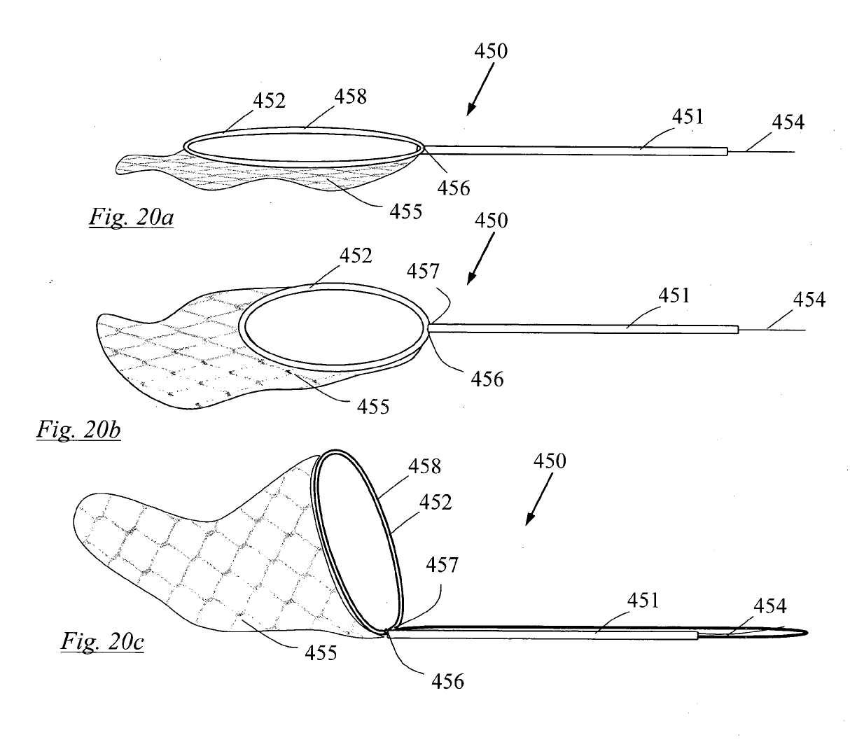

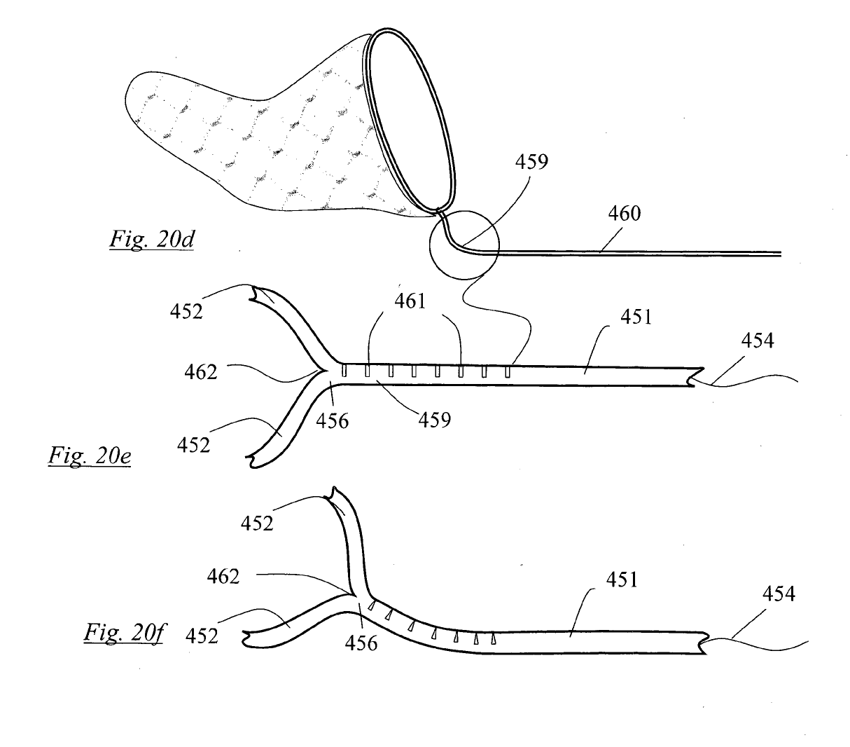

Referring now to FIG. 20a to FIG. 20c there is shown yet another embodiment of the invention. In this case a basket assembly 450 comprises a support member 451, an engagement ring 452, a pull cable 454 and a net 454. The support ring 452 comprises two struts 458 and the net is attached to said struts 458. The support member comprises a lumen and the pull cable extends through the lumen of the support member 451. The basket assembly 450 comprises a collapsed delivery configuration as shown in FIG. 20a, an expanded configuration and an expanded articulated configuration as shown in FIG. 20c. The ring member is comprised of Nitinol and has a remembered expanded configuration. The remembered expanded configuration of the engagement ring 452 comprises a substantially planar hoop and the plane of the hoop is aligned with the axis of the elongate support member 451. The basket assembly 450 further comprises a junction 456 where the support member 451 and the engagement ring 452 are interconnected.

With reference to FIG. 20c, the basket assembly further comprises an articulation region 457 adjacent to the junction 456. Pulling the pull cable 454 relative to the support member 451 causes the ring member to articulate relative to the support member and the ring member 452 makes an angle with the support member 451. In one embodiment the angle is less than 90°. In another embodiment the angle is equal to or greater than 90°. In one embodiment the articulation region 457 is adjacent the junction 456. In one embodiment the articulation region forms part of the engagement ring 452 adjacent the junction 456. In another embodiment the junction 456 comprises the articulation region 457.

Yet another embodiment is shown in FIG. 20d to FIG. 20f where the articulation region 457 forms part of the distal end 459 of the support member 451 adjacent the junction 456. With this embodiment activation of the pull cable 454 causes the distal end 459 of the support member 451 to change shape and this results in the engagement ring 452 articulating relative to the proximal end 460 of the support member 451. The shape change of the distal end 459 of the support member 451 comprises a change from a straight configuration to a curved configuration. The curved segment at the distal end 459 of the support member 451 has the effect of displaces and angulates the ring member 452 relative to the proximal end of the support member 451 and this allows the basket to reach around curved segments. This feature is especially useful at bifurcations where the occlusion needs to be removed from two branches simultaneously.

FIG. 20e and FIG. 20f show close up views of one embodiment of articulating basket assembly described in FIG. 20d. With this embodiment the support member 451 comprises a tube and the distal end 459 of said tube comprises a slotted section. The slots 461 in the slotted section are partial slots that extend around a portion of the diameter. In the embodiment shown the slots are all on one side of the tubular member 451. The side of the tubular member 451 with the slots 461 is more compressible than the side of the tube that possesses no slots. The pull cable is connected to the tubular member distal of the slotted section. In one embodiment the cable 454 is fixed to the support member 451. In another embodiment the cable is fixed to the junction 456. In another embodiment the cable 454 is fixed to the ring member 452. In yet another embodiment the slotted section 459 of the support member 451 comprises an exit port and the cable 454 extends through the exit port and is fixed to the engagement ring 452.

When the pull cable 454 is activated (tensioned) it applies a compressive force on the support member 451. Since the distal end 459 of the support member 451 has a compressible section this section compresses under the force. The compression of the distal section 459 comprises the closing of the slots in the tubing and this is shown in FIG. 20f. The compression of the slots 461 causes the distal section of the support member to change shape. In the embodiment shown in FIG. 20f the deformed shape comprises a curved segment at the distal end 459 of the support member 451. It will be appreciated that curves of tighter radius can be achieved by increasing the number of slots in the support member.

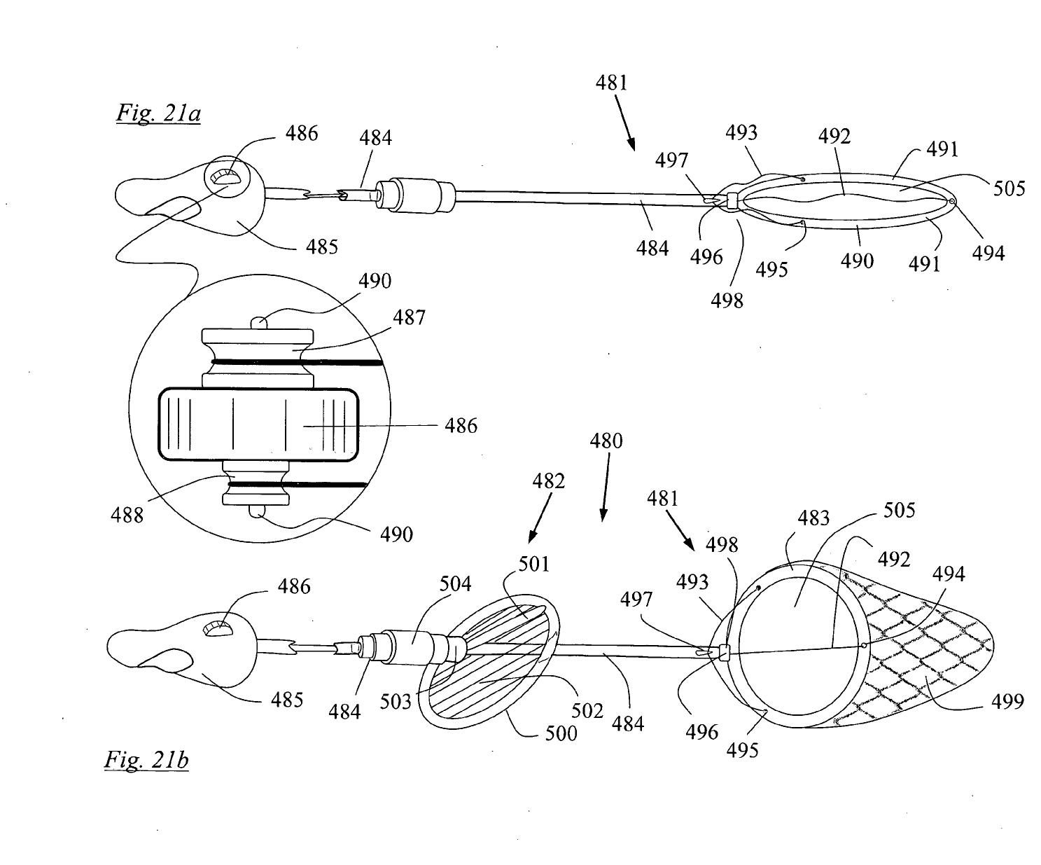

With reference to FIG. 21a and FIG. 21b another device 480 of the invention is described. The device 480 comprises a basket assembly 481, and a debonder assembly 482. The basket assembly 481 comprises a frame 483 a support member 484 and a handle 485. The support member comprises an elongate tube and the frame 483 is fixed to the distal end of the support tube 484. The frame comprises a collapsed configuration for delivery through the vasculature and an expanded configuration. The frame further comprises a clot engagement opening 505 and in the expanded configuration the clot engagement opening is sized such that the frame can engage the outer circumference of the clot and such that the clot can be forced through the opening and into the capture net 499. In the collapsed state the engagement opening 505 is substantially closed as is shown in FIG. 21a. The frame expanded state further comprises an angulation of the frame 483 with respect to the support member 484. In the collapsed state the axis of the frame lies substantially parallel to the axis of the support member 484. For the purposes of FIG. 21a-b the axis of the frame 483 shall mean a line drawn between the exit port 496 at the distal end of the support member 484 and the attachment point 494 on the frame 483. In the expanded state the axis of the frame is subtended at an angle to the axis of the support member. In one embodiment the axis of the frame 483 makes an angle of 30° or greater with the axis of the support member 484. In another embodiment the axis of the frame 483 makes an angle of 45° or greater with the axis of the support member 484. In yet another embodiment the axis of the frame 483 makes an angle of 60° or greater with the axis of the support member 484. In yet another embodiment the axis of the frame 483 makes an angle of 90° or greater with the axis of the support member 484.

The basket assembly 481 further comprises a first cable 492 extending from the handle 485 through the lumen of the support member 484, exiting the support member 484 at first exit port 496 and attaching to the frame 483 at first cable attachment 494. The first cable 492 when tensioned is responsible for expanding the ring member 490. It will be appreciated that the strut 491 width and thickness dimensions can be adjusted along the length of the strut 491 so as to assist in opening the ring member 490. The proximal end of the first cable 492 is fixed to an activation mechanism in the handle 485. In one embodiment the activation mechanism comprises a thumb wheel 486, and a first spool 487 fixed to the thumbwheel 486. A portion of the thumbwheel 486 extends through the wall of the handle 485 and allows the user to rotate the thumbwheel 486. The spool 487 is configured such that rotation of the thumbwheel 486 causes the first cable 492 to be wound onto the spool 487. The diameter of the first spool 487 controls the rate at which the first cable 492 is wound and thus the rate at which the engagement opening 505 of the ring member 490 is expanded.

The basket assembly 481 further comprises a second cable 493 extending from the handle 485 through the lumen of the support member 484, exiting the support member 484 at the second exit port 497 and attaching to the frame 483 at second cable attachment points 495. In one embodiment the second cable comprises two cables and the second attachment point comprises two attachment points. The second cable 493 when tensioned is responsible for angulating the ring member 490 with respect to the distal end of the support member 484. The basket assembly 481 comprises an articulation region adjacent the end of the support member. In one embodiment the frame 483 comprises an articulation region adjacent the junction 498 between the frame 483 and the support member 484. In another embodiment the distal end of the support member 484 comprises an articulation region. The proximal end of the second cable 493 is also fixed to an activation mechanism in the handle 485. In one embodiment the activation mechanism comprises the thumb wheel 486, and a second spool 488 fixed to the thumbwheel 486. The second spool 488 is configured such that rotation of the thumbwheel 486 causes the second cable 493 to be wound onto the spool 488. The diameter of the second spool 488 controls the rate at which the second cable 493 is wound and thus the rate at which the angulation of the engagement ring 490 is changed with respect to the support member 484. FIG. 21b shows a representation of the thumbwheel 486 wherein the first spool 487 and the second spool 488 are integral with the thumbwheel. The diameter of the first spool 487 and the second spool 488 can be independently adjusted so as to balance the rate of ring member 490 opening with the rate of angulation of the frame 483. It will also be appreciated that the first spool 487 and the second spool 488 may be mounted on separate thumbwheels. In this case the handle 485 would comprise two thumbwheels.

The device further comprises a net 499 mounted to the ring member as previously described. In one embodiment the device comprises a clot debonder 482. The clot debonder is as shown in FIG. 21c and comprises a debonding ring 500, a connector strut 501, an elongate tube 503 and a hub 504. The debonding ring 500 further comprises a lased surface 502 and said lased surface 502 comprises filaments lased across the opening defined by the debonder ring 500. The debonding ring 500 is connected to the tubular member 503 and the tubular member 503 extends proximally to control hub 504. The elongate tube 503 is configured to slide over the support member 484 such that the lased surface 502 can be advanced to engage with the occlusion.

A significant advantage of the device 480 of this invention is that the frame 483 assumes the collapsed configuration when not activated. This means that it can be advanced through the lumen of a microcatheter without any restraint and that it applies no radial force to the wall of the microcatheter. This allows the device to be constructed with a very low profile and allows the frame 483 to be advanced through the lumen of a microcatheter with ease. Neurovascular vessels are highly tortuous and ease of advancement is key to delivering the device to the target vessel segment.

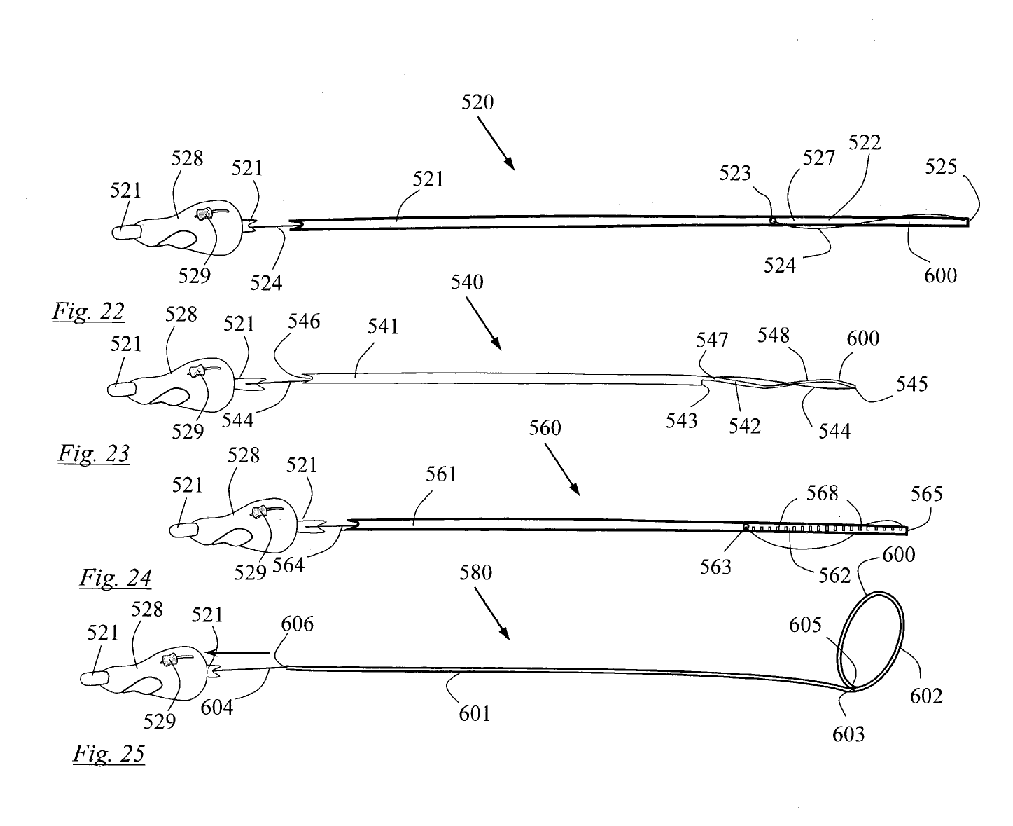

Another aspect of the invention is shown in FIG. 22 through to FIG. 30 where a variety of debonder assemblies are disclosed. These debonder assemblies generally comprise a distal section wherein said distal section comprises an elongate member in the delivery configuration and the elongate member undergoes a shape change to form a ring member under the influence of a pull cable and said ring member is employed in conjunction with baskets to break the bonds that exist between occlusive clot and the wall of a vessel.

FIG. 22 shows a debonder assembly comprising an elongate tube 521 and a pull cable 524. The elongate tube further comprises a distal section 522 an exit port 523 a distal cable attachment 525 and an articulation region 527. The pull cable 524 and the elongate tube extend proximally to a handle 528. The pull cable 524 is connected to an activator 529 in the handle 528 and said activator 529 is configured to allow the user to tension the pull cable 524. In one embodiment the activator 529 comprises a slider. In another embodiment the activator 529 comprises a thumbscrew. In one embodiment the proximal end of the elongate tube 521 is fixed to the distal end of the handle 528 and the pull cable exits the elongate tube via its proximal lumen. In another embodiment the elongate tubing extends through the handle and provides a continuous lumen through the handle such that the assembly can be interfaced with other devices. With this embodiment the proximal end of the elongate tube 521 comprises a proximal exit port 531 and the cable 524 exits the lumen of the elongate tube 521 through the proximal exit port. The proximal exit port 531 is preferably distal of the activator 529. The distal end of the cable 524 is fixed at attachment point 525 at the distal end of the distal section 522. The assembly is delivered to the treatment site in the collapsed configuration as shown in FIG. 22 with the pull cable relaxed. In a preferred embodiment the pull cable 524 encircles the distal section 522 once between the exit port 523 and the attachment point 525. At the treatment site the distal section is transformed into a ring member 600 as shown in FIG. 25. The ring member comprises a generally circular or elliptical hoop and is configured to abut a vessel occlusion. The ring member 600 preferably engages the occlusion adjacent the interface between the occlusion and the vessel wall. In this way the ring member 600 delivers an abutment force to the interface between the clot and the vessel wall and this is the region where clot separation is most desired. The device further comprises an articulation region 527 adjacent the exit port 523. In one embodiment the articulation region comprises a local weakening of the tube in that region. In another embodiment the articulation region comprises at least one cut or slot in the wall of the tube in the articulation region. In another embodiment the cut comprises a spiral cut. In another embodiment the cut comprises a circular cut. In another embodiment the cut comprises at least one helical cut. In another embodiment the cut comprises a cut thickness. In another the cut thickness comprises at least two cut thicknesses. In yet another embodiment the articulation region comprises a plurality of patterned slots.

FIG. 23 shows a debonder assembly 540 which is similar to the assembly shown in FIG. 22 and comprises an elongate tube 541 and a pull cable 544. The elongate tube 541 further comprises a distal section 542 a lumen 546, a lumen distal end 543 a distal cable attachment 545 and an articulation region 547. The pull cable 544 and the elongate tube extend proximally to a handle 528. The construction and functions of the handle 528 are the same as was described in FIG. 22.

The lumen 546 extends from its distal end 543 to the proximal end of the handle 528 and is sized so as to accommodate the pull cable 544 and another elongate assembly such as a basket assembly. The debonder assembly 540 is delivered to the treatment site in the collapsed configuration as shown in FIG. 23 with the pull cable 544 relaxed. At the treatment site the distal section 542 is transformed into a ring member 600 as shown in FIG. 25. The distal section 542 comprises a strut 548. The strut 548 preferably comprises a spiral member. In one embodiment the strut 548 is cut from a hypotube and the spiral extends the entire length of the strut. In one embodiment the spiral comprises a 360° spiral. The pull cable extends from the lumen distal end 543 and is attached at attachment point 545. In a preferred embodiment the pull cable encircles the distal section 542 once between the lumen distal end 543 and the attachment point 545. When the pull cable is activated the distal attachment moves towards the lumen distal end 543. The strut progressively forms into a hoop and articulates about the articulation region 547.

The ring member 600 when formed comprises a generally circular or elliptical hoop and is configured to abut a vessel occlusion. The ring member 600 preferably engages the occlusion adjacent the interface between the occlusion and the vessel wall. The assembly 540 further comprises an articulation region 547 adjacent the distal end 543 of lumen 546. In one embodiment the articulation region 547 comprises a local weakening of the strut 548 in that region. In another embodiment the articulation region 547 comprises at least one cut or slot in the wall of the strut in the distal section 542.

FIG. 24 shows a debonder assembly 560 which is similar to the assembly shown in FIG. 22 and comprises an elongate tube 561 and a pull cable 564. The only difference between the device 520 of FIG. 22 and the debonder assembly 560 of FIG. 24 lies in the construction of the distal section 562. The distal section 562 comprises a plurality of slots 568 arranged along the substantially the entire length of the distal section. The plurality of slots 568 are arranged in a helical pattern. Each slot has a significant circumferential component and each slot comprises a width. In one embodiment the circumferential component of the slot comprises at least one quadrant. The width of the slot 568 is configured so as to facilitate compression of the slot 568 by the pull cable 564. Preferably the sum of the widths of all the slots 568 in the distal section 562 should add up to a dimension that is less than the difference between the inner and outer circumference of the ring member 600 when the ring member 600 sized to the diameter of the target vessel. In a preferred embodiment the pull cable 564 encircles the distal section 562 once between the exit port 563 and the attachment point 565.

FIG. 25 shows a debonder assembly 580 with the distal section formed into a hoop shaped ring member 600. The debonder assembly 580 represents the expanded configuration of the debonder assemblies 520, 540 and 560 of FIG. 22-24. The assembly comprises a ring member 600, a support member 601, a support member lumen 606, a distal section 602, a cable exit (port/lumen) 603, a pull cable 604, a handle assembly 528 (not shown), and a distal attachment 605.

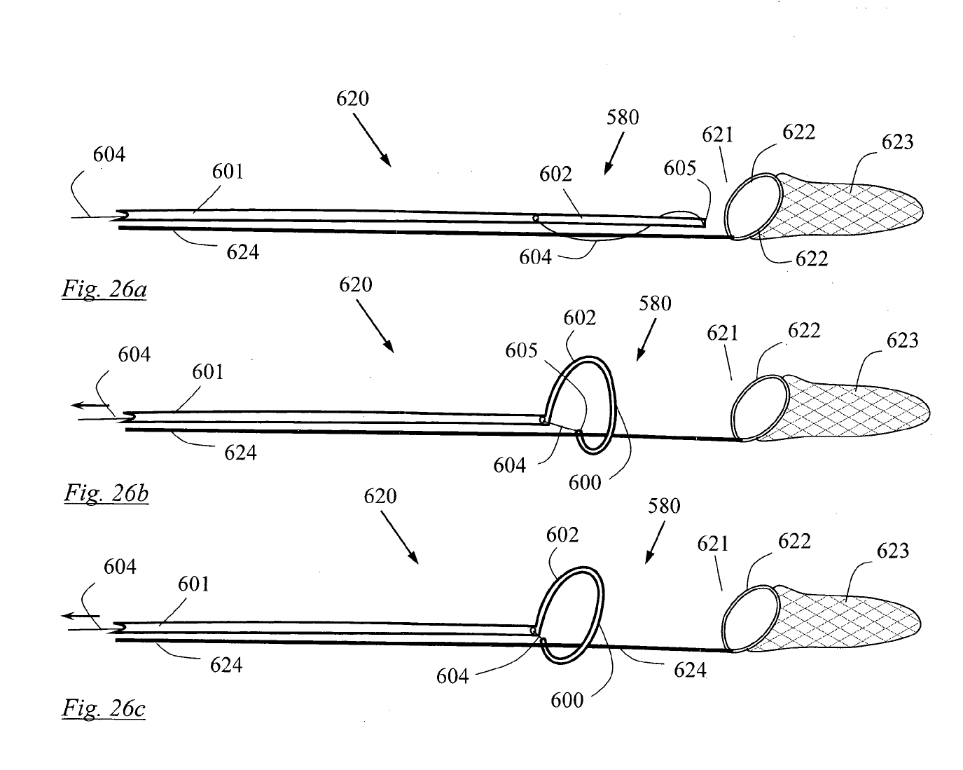

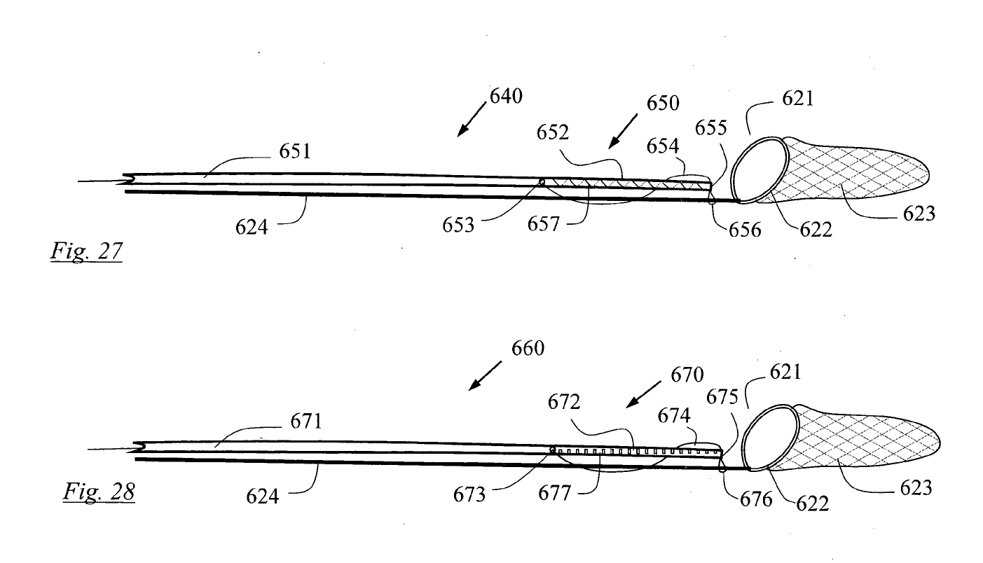

FIG. 26a to FIG. 26c shows the arrangement of a device 620 wherein a debonder assembly 580 as described in FIG. 22-25 is being used in conjunction with a basket assembly 621. The basket assembly 621 comprises a hoop frame 622, a net 623 and a support member 624. The frame 622 is similar to the frame construction employed in FIG. 6. The support member 624 of the basket assembly 621 extends parallel to the elongate tube 601 of the debonder assembly 580. The support member 624 extends between the pull cable 604 and the distal section 602 of the debonder assembly 580. In one embodiment both the support member and the elongate tube extend through the lumen of a microcatheter. In another embodiment the support member extends through the lumen of the elongate tube and the elongate tube is configured to be advanced or retracted relative to the support member. In FIG. 26a the basket assembly 621 is shown in the expanded configuration with the debonder assembly in the collapsed configuration. In FIG. 26b the pull cable 604 is activated and the distal section 602 is being reshaped into an engagement ring 600. In FIG. 26c the pull cable 604 is activated further through the handle 528 (not shown) and the reshaping of the distal section 602 is almost complete. It will be appreciated that the engagement ring 600 engages the occlusion in an area adjacent the wall of the vessel and that some clot may project through the opening in the engagement ring 600. However the primary purpose of the engagement ring 600 is to provide a reaction force to the action of the basket assembly 621. This reaction force allows the basket assembly to be retracted strongly without fear of vessel rupture or dissection.

Using the device 620 of the invention comprises at least some of the following steps:
Advancing a guide catheter into a large diameter vessel proximal of the cerebral vasculature (the CCA or the ICA).
Advancing a microcatheter through the lumen of the guide catheter until its distal end is advanced distal of the tip of the guide catheter.
Further advancing the microcatheter with the assistance of a guidewire within the lumen of the microcatheter.
Manipulating both the microcatheter and the guidewire until the tip of the microcatheter is across the occlusive clot.
Withdrawing the guidewire 423 from the lumen of the microcatheter.
Advancing the device 620 through the lumen of the microcatheter with both the basket and the clot debonder in the collapsed configuration.
Deploying the basket from the microcatheter distal of the occlusion.
Expanding the frame of the basket distal of the occlusion.
Retracting the microcatheter and exposing the distal section 602 of the debonder assembly 580.
Activating the cable such that the distal section 602 of the debonder assembly changes shape and forms an engagement ring.
Engaging the engagement ring with the proximal end of the clot.
Engaging the hoop frame 622 of the basket assembly 621 with the distal face of the clot.
Retracting the basket assembly while holding the debonder assembly 580 stationary.
Disengaging the clot from the vessel wall.
Applying a capture force to the clot.
Forcing the clot into the capture opening of the basket.
Disengaging the engagement ring 600 from the proximal face of the occlusion.
The step of disengaging the engagement ring 600 comprises at least removing some of the tension in the cable such that the engagement ring 600 at least partially reverts to its original configuration.

FIG. 27 shows a device 640 which comprises a basket assembly 621 and a debonder assembly 650. The basket assembly 621 is as described in FIG. 26a-c. The debonder assembly 650 comprises an elongate tube 651, a distal section 652, a cable 654, a cable exit port 653 and a cable attachment 655. The distal section 652 comprises a tube and the tube comprises a spiral cut. The spiral cut allows the distal section 652 to deform into a ring member for clot abutment when the cable 654 is tensioned. The distal section 652 further comprises a cable loop 656. The cable loop 656 comprises a loop of yarn wherein both ends of the yarn are fixed to the distal section 652. The loop 656 is sized to accommodate the support member 624 of the basket assembly 621 and the loop 656 holds at least a portion of the support member 624 adjacent the distal section 652. When the distal section 652 is expanded and assumes its expanded configuration as a ring member the loop guides the ring member along the support member 624 as it is advanced and retracted.

FIG. 28 shows a device 660 which comprises a basket assembly 621 and a debonder assembly 670. The debonder assembly 670 is very similar to the debonder assembly 650 of FIG. 27. The debonder assembly 670 comprises an elongate tube 671, a distal section 672, a cable 674, a cable exit port 673 and a cable attachment 675. The distal section 672 comprises a tube and the tube comprises a plurality of slots. The plurality of slots 677 controls the bending of the distal section 652 when the cable 654 is tensioned. The slots 677 are arranged such that the distal section deforms into a hoop and said hoop is articulated so as to create a distal abutment ring for clot engagement. The distal section 672 further comprises a cable loop 676 and the cable loop 676 functions in the same manner as cable loop 656 of FIG. 27.

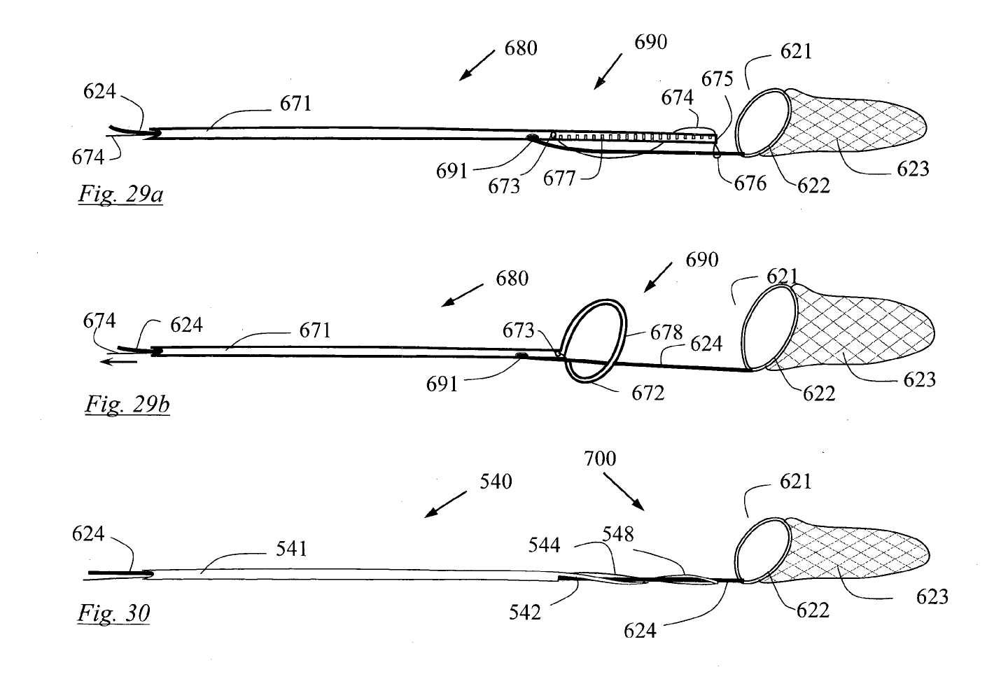

The device 680 of FIG. 29a and FIG. 29b comprises a basket assembly 621 and a debonder assembly 690. The debonder assembly 690 comprises an elongate tube 671, a distal section 672, a cable 674, a cable exit port 673 and a cable attachment 675. The distal section 672 comprises a tube and the tube comprises a plurality of slots 677. The plurality of slots 677 controls the bending of the distal section 652 when the cable 654 is tensioned. The slots 677 are arranged such that the distal section deforms into a hoop and said hoop is articulated so as to create a distal abutment ring for clot engagement. The distal section 672 further comprises a cable loop 676 and the cable loop 676 functions in the same manner as cable loop 656 of FIG. 27. The elongate tube 671 comprises an inner lumen 691 and said inner lumen is sized to accommodate the support member 624 over at least a portion of the length of the elongate tube 671. The elongate tube 671 further comprises an inlet 691 and said inlet 691 is sized to allow the support member 624 access the lumen of the catheter. In one embodiment the inlet 691 is located proximal of the cable exit port 673. In another embodiment the inlet 691 is distal of the exit port 653. In another embodiment the inlet 691 is adjacent the exit port 653. In yet another embodiment the exit port 653 comprises the inlet 691.

In yet another embodiment the shape change of the distal section 672 is achieved using two cables. The first cable is attached to the distal section at the distal end of the distal section. This cable when activated pulls the attachment 676 towards the exit port 673 and thus forms an engagement ring 678. The second cable is attached to the distal section 672 proximal of the distal end and causes the ring to articulate such that the ring 678 comprises a distally facing abutment ring. In one embodiment the first and second cables are activated with a single activator 529. In another embodiment the first and second thumbwheels are activated by two separate thumbwheels.

The device 700 of FIG. 30 comprises the basket assembly 621 and the debonder assembly 540 both of which have been described previously. The figure shows the two assemblies configured as a device for use in treating acute occlusions. The strut 548 of the distal section 542 is wrapped around the support member 651 of the basket assembly 621 in the delivery configuration.

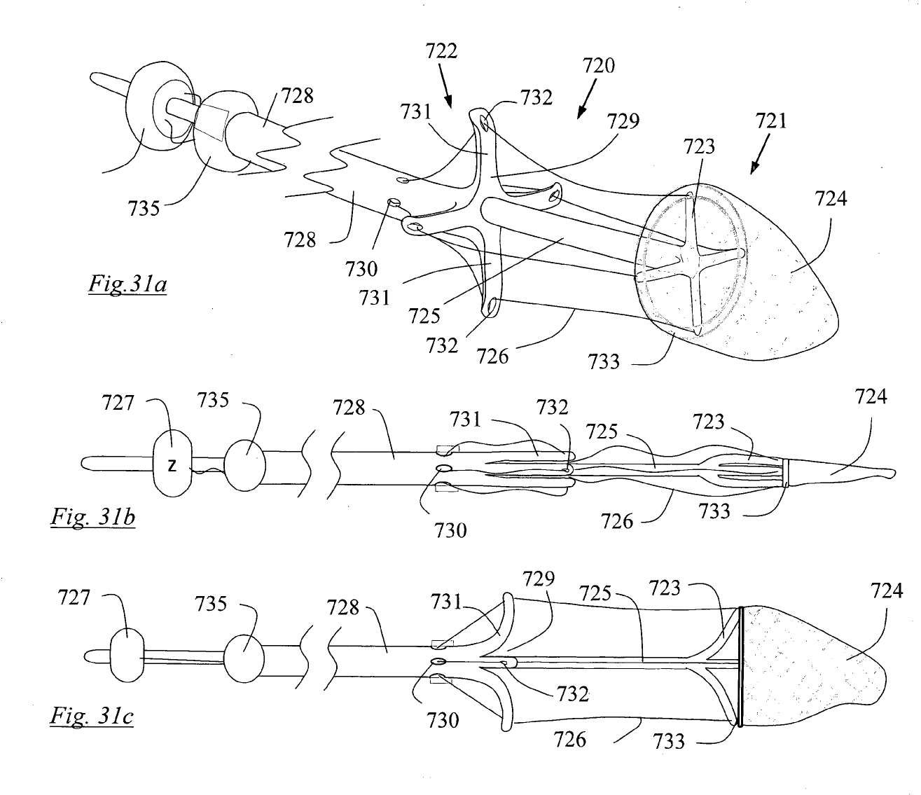

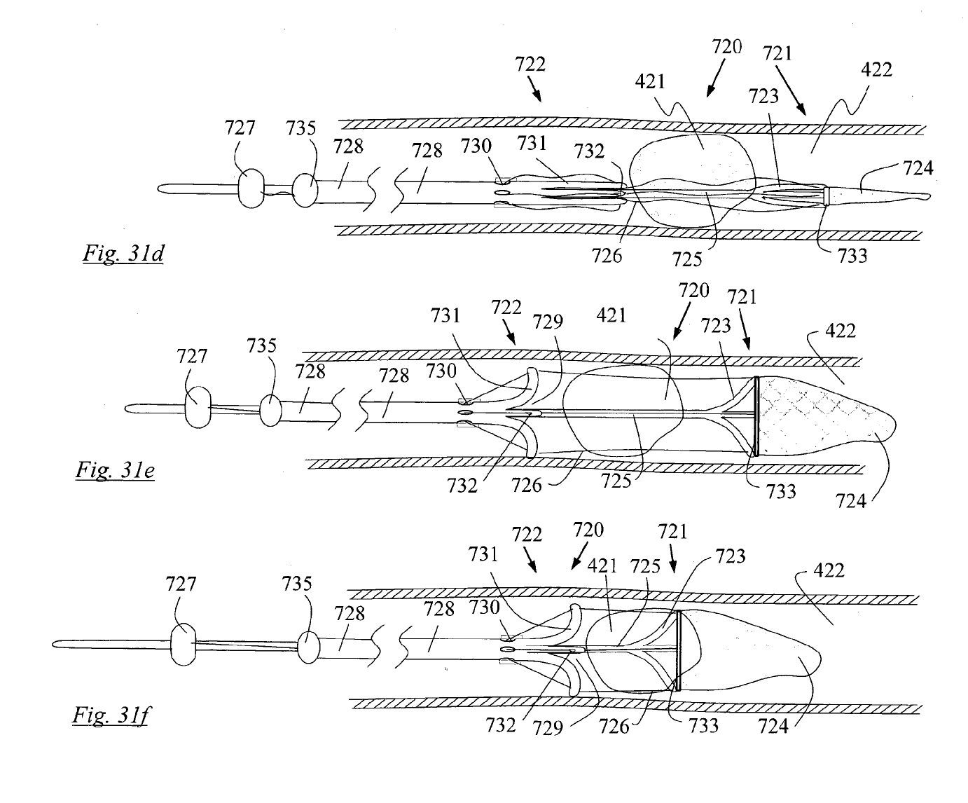

FIG. 31a-31c show another device 720 of the invention. The device comprises a debonder assembly 722 and a basket assembly 721. The basket assembly comprises support struts 723 and a net 724. The support struts 723 are connected to the user end at the basket proximal hub 727 via a plurality of pull cables 726. The pull cables 726 are spaced apart from the support member 725. The pull cables are assembled through cable guides 732 of the struts 731 in the debonder assembly 722. In this embodiment the debonder assembly 722 and basket assembly 721 move relative to each other in order to retrieve a vessel obstruction while the pull cables 726 remain adjacent the vessel wall.

FIG. 31b is an elevation view of the device of FIG. 31a in the collapsed configuration and FIG. 31b shows the device of FIG. 31a in an expanded configuration. The support member 225 is assembled inside elongate tube 728. The support struts 731 are connected to elongate type 728. The elongate tube contains a plurality of exit ports 730. In this embodiment the pull cables are fixed to the support struts 723 at the distal end and the basket proximal hub 727, and are moveable through the cable guides 732 and exit ports 730.

FIG. 31c shows the device in a partially activated state with the support struts 723 and struts 731 expanded. The device may be partially actuated by mobbing the proximal basket hum 727 relative to the debonder hub 735.

FIG. 31d-FIG. 31f shows the device of FIG. 31a inside a vessel removing an occlusive clot 421. FIG. 31d shows the device with the basket assembly 721 distal of a clot and the debonder assembly 722 proximal of the clot. FIG. 31e shows the device 720 in a partially actuated state with the struts 731 and support struts 723 in an expanded configuration. In FIG. 31f the basked assembly is located adjacent the occlusive clot and the pull cables 726 are in tension. This draws the basket assembly 721 and the debonder assembly 722 together. The engagement segment 729 of the debonder assembly provides a support surface for the clot as the basket assembly 721 engulfs the occlusive clot.

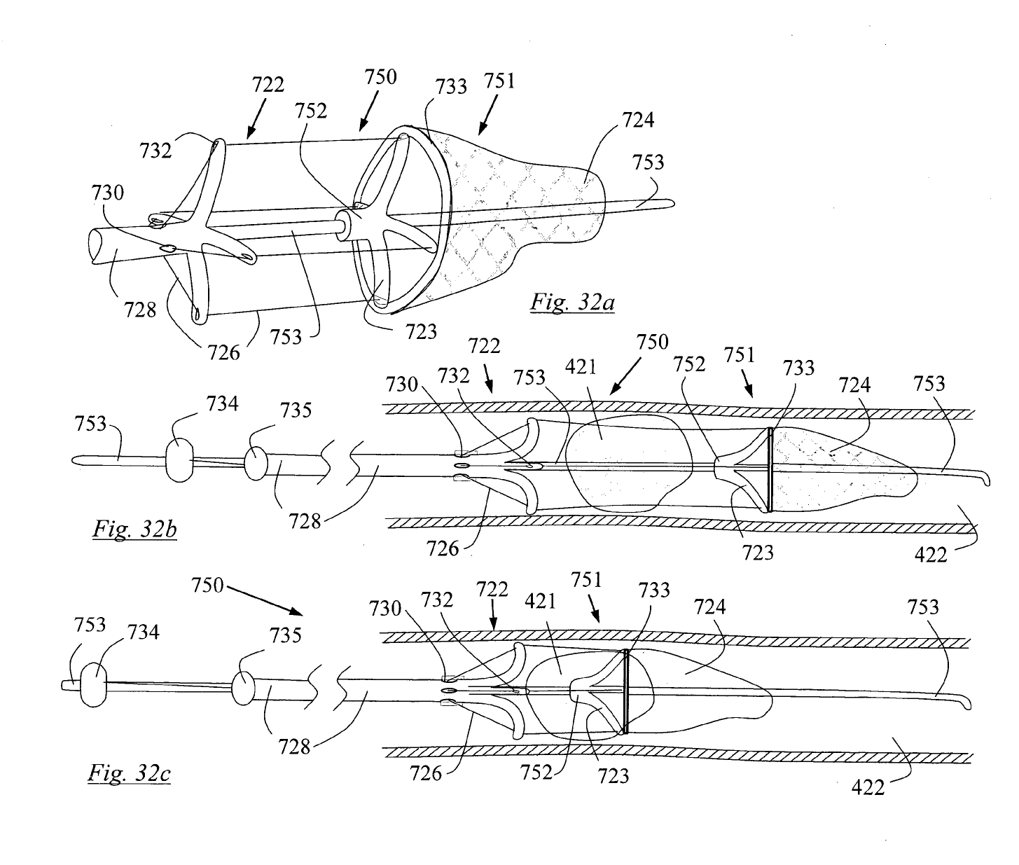

FIG. 32a-FIG. 32c show another device 750 of the invention. In FIG. 32a the debonder assembly 722 is fixed to an elongate tube 728 similar to the device of FIG. 31a but in this device the basket assembly 751 is slidable relative to a guidewire 753. FIG. 32b shows the device 750 in a vessel with an occlusive clot 421 with the basket assembly deployed distal of the occlusive clot. In FIG. 32c the control hub 734 is moved relative to the debonder hub 735 to draw the basket assembly 751 over the clot. The guidewire 753 is movable relative to the debonder assembly 722 and the basket assembly 751. With this device the user may leave the guidewire 753 in place after removing the basket assembly 751 containing the occlusive clot 421.

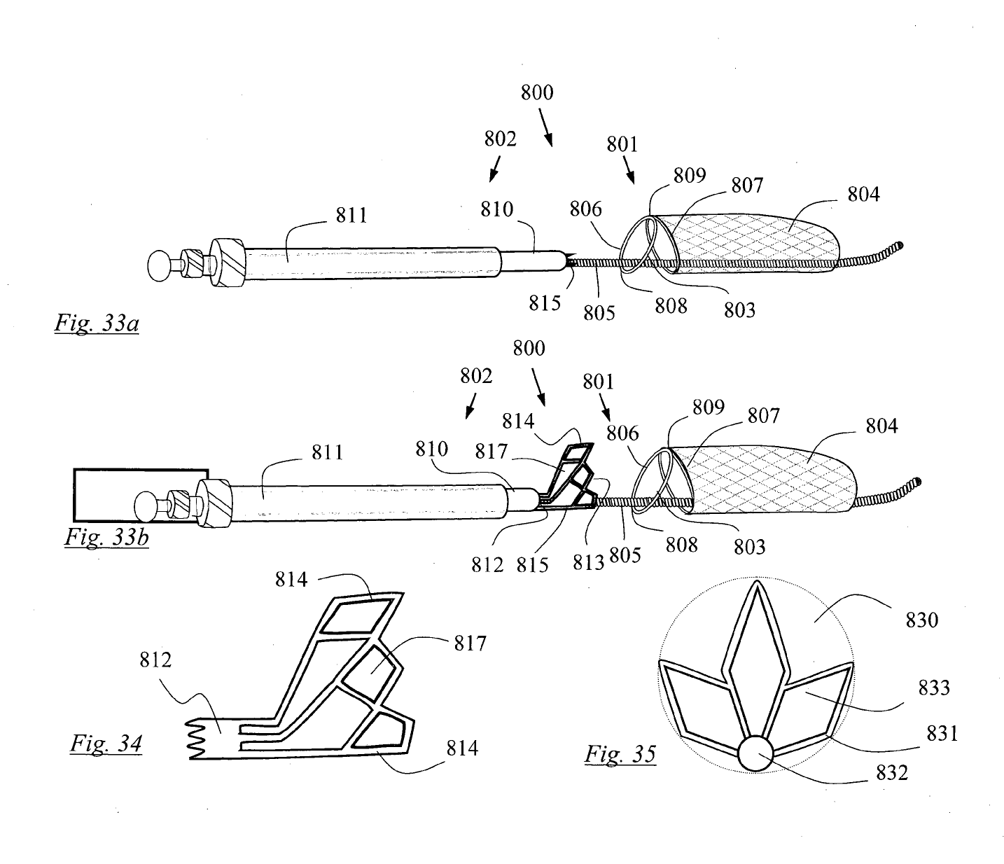

FIG. 33a and FIG. 33b show another device 800 of the invention. In this case the device 800 comprises an eccentric basket 801 and an eccentric debonder 802. The eccentric basket 801 comprises a frame 803, a net 804 and a support member 805. The frame comprises a proximal hoop 806, a distal hoop 807 and an expansion member 809. The frame expansion comprises of two components. Firstly the proximal and distal hoops form. The proximal and distal hoops comprise pairs of struts in the collapsed configuration. The struts move apart to form a hoop when the external restraint is removed. Secondly the first and second hoops undergo an angular displacement with respect to each other. This angular displacement is driven by elastic energy stored in the expansion member 809. The expansion member 809 also interconnects the proximal hoop 806 to the distal hoop 807. When the frame 803 is in the expanded configuration the expansion member 809 provides the frame 803 with a significant portion of its resistance to collapse.

Thus the expansion member 809 is configured to withstand significant strain and provide good resistance to collapse. Preferably the expansion member 809 comprises a metal. More preferably the metal comprises a nitinol. Preferably the expansion member 809 and the frame 803 comprise the same material and preferably the expansion member 809 and the frame 803 are integral. In one embodiment the expansion member 809 comprises a strut connecting the proximal hoop 806 to the distal hoop 807. In one embodiment the strut comprises a width and a thickness and the ratio of the width and the thickness comprises the aspect ratio of the strut. Preferably the aspect ratio of the strut is greater than 1. More preferably the aspect ratio is 1.5 or greater.

The frame 803 is mounted to the support member 805 at attachment 808. Preferably the attachment 808 comprises an attachment between the proximal hoop 806 and the support member 805. In one embodiment the net 804 is attached to the distal hoop 807. In another embodiment the net 804 is attached to the proximal hoop 806.

The debonder 802 comprises an elongate tube 812, a control handle 816 and a debonding element 815. The debonding element 815 comprises a plurality of engagement struts 814 and the engagement struts are configured so as to create the clot engagement face 813 when expanded. In one embodiment the debonding element or clot engager 815 comprises a plurality of struts forming a first section and a second section, with the first section tapering outward and distally from elongate member 812 and connected to the second section, and the second section comprising a plurality of cells defined by a plurality of struts and arranged around at least a portion of the circumference of an axis substantially parallel to that of the elongate member. In another embodiment these cells are arranged around the entire circumference of said axis.

The debonding element 815 comprises a plurality of cells 817 wherein each cell is defined by a plurality of boundary struts 814. It will be appreciated that a number of cell 817 and strut 814 arrangements are possible in creating a clot engagement surface 813.

The debonding element is connected to the elongate tube 812 and the debonding element 815 is advanced or retracted using the control handle 816 at the user interface 817. The user interface comprises the proximal hub 818 of the guide catheter 811, the proximal hub 819 of the microcatheter 810, the control handle 816 of the debonder 802 and the proximal end of the support member 805. The guide catheter hub 818 and the microcatheter hub 819 both comprise luer connectors and both facilitate the addition of accessories such as Y-connectors, Touhy Borsts and syringes. These accessories facilitate flushing as well as locking the guide catheter to the microcatheter 810 or locking the microcatheter 810 to the elongate tube 812. In one embodiment the control handle 816 comprises a luer fitting. In another embodiment the control handle 816 comprises a locking element for locking the control handle to the support member 805.

FIG. 34 shows a blown up view of one embodiment of the clot debonding element 815 of FIG. 33. In the embodiment shown the struts 814 are integral with the distal end of the elongate tube 812. In another embodiment the clot debonding or clot engaging element 815 may be a separate component to elongate member 812.

FIG. 35 shows an end view of another eccentric debonding element 830. The debonding element 830 comprises a plurality of cells 833 defined by a plurality of struts 831. The plurality of cells 833 are configured such that in the expanded state the debonding element 830 will engage with a substantial portion of a vessel with a substantially circular cross-section. The struts 831 are connected to an elongate tube 832.

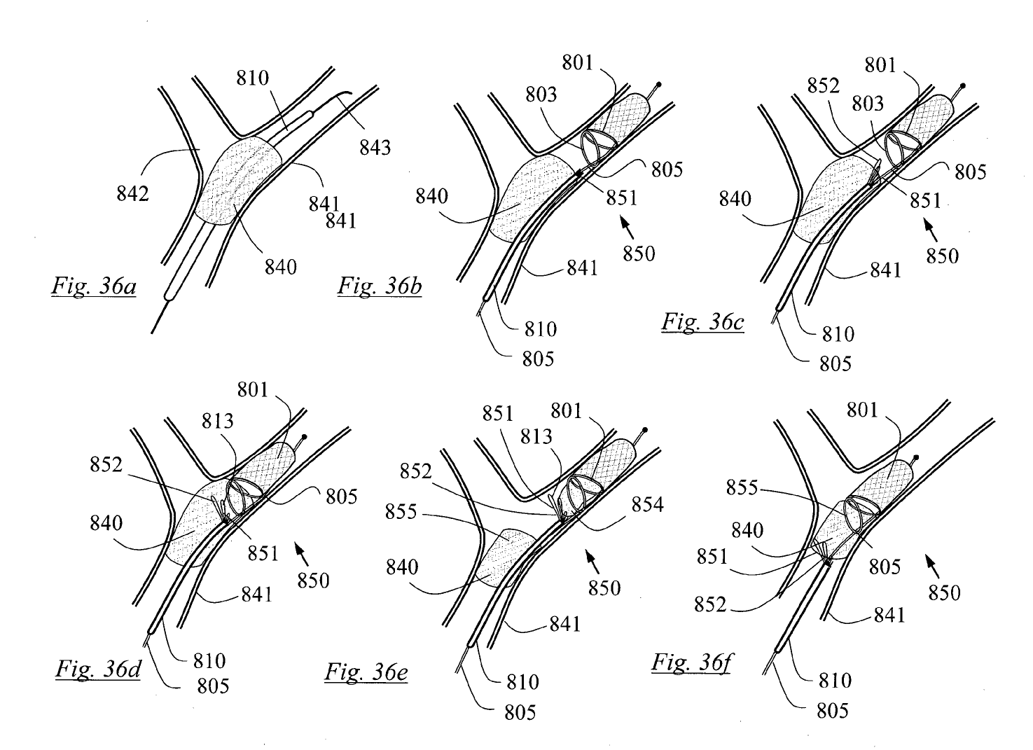

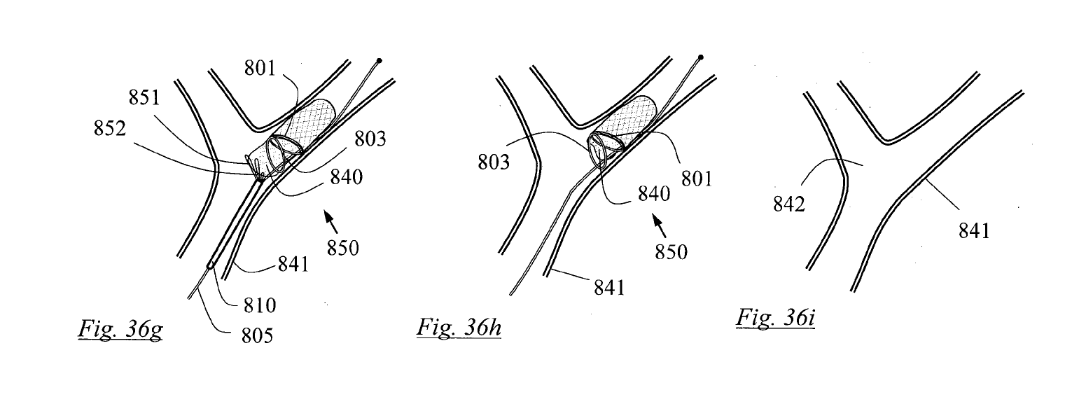

Now with reference to FIG. 36a to FIG. 36i there is shown another device 850 which is very similar to the device describe in FIG. 33 to FIG. 35 and similar numerals will be employed to describe similar elements. FIG. 36a to FIG. 36i show the procedural steps associated with using the device 850. The device 850 comprises a basket 801 and a debonder 851. The debonder 851 is an eccentric debonder and is similar to the debonder 802 described with reference to FIG. 33. The debonder 851 comprises a debonding element 852 and an elongate tube 812. The elongate tube 812 comprises a lumen sized to accommodate the support member 805 of the basket 801. The debonding element 852 is fixed to the distal end of the elongate tube 812 and comprises a collapsed state for delivery, a deployed partially expanded state and a fully deployed state. The debonder element 852 comprises a plurality of struts 814 and said struts 814 are configured to expand on deployment. The engagement struts 814 are configured so as to create a clot engagement surface 853 when expanded. The debonding element 852 comprises a plurality of cells 817 wherein each cell is defined by a plurality of boundary struts 814. It will be appreciated that a number of arrangements of cells 817 and struts 814 are possible in creating a clot engagement surface 853.

The debonding element 852 deployed state comprises an intermediate diameter when expanded in an unconstrained fashion. Preferably the debonding element 852 comprises a nitinol, a shape memory or a super elastic material. The debonding element 852 comprises an engagement surface 853 in the deployed state. The engagement surface 853 is a distally facing surface and is configured to engage with the occlusion 840. The engagement surface 813 comprises a tapered surface and when the tapered surface engages with the clot 840 the reaction force of the clot 840 causes the debonding element 852 to expand further. In one embodiment the further expansion of the debonding element 852 comprises an articulation of at least a portion of the engagement surface 853. In another embodiment the further expansion comprises a change in shape of the cells 817 of the debonding element 852. Conversely, when the debonding element 852 is disengaged from the occlusion 840 the debonding element 852 partially collapses, returning to its biased partially expanded configuration. Furthermore, when the debonding element is withdrawn through an occlusion 840 or a partial occlusion the outer side of the tapered surface engages with the clot and the debonding element 852 is further collapsed by the reaction force of the occlusion 840 on its outer surface. Thus the debonding element 852 spontaneously engages when advanced against an occlusion 840 and collapses when retracted through a restriction or occlusion 840.

FIG. 36a shows the start of the procedure and the steps for gaining access to the distal side of the occlusion. The steps comprise:
Advancing a guide catheter 811 into a large supra aortic vessel.
Advancing a guidewire 843 and a microcatheter 810 through the lumen of the guide catheter 811.
Manipulating the guidewire 843 and the microcatheter in concert so as to access the target vessel.
Passing the guidewire 843 and the tip of the microcatheter 810 across the occlusion 840.
Removing the guidewire 843 from the lumen of the microcatheter.

With reference to FIG. 36b, the device 850 is advanced through the lumen of the microcatheter 810. The procedural steps involved in delivering and deploying the basket 801 comprise:
Providing the device with the basket 801 and the debonder 851 in the collapsed configuration.
Inserting the distal end of the device 850 into the lumen of the microcatheter 810.
Advancing the device 850 through the lumen of the microcatheter 810 until the basket frame 803 exits the microcatheter 810.
Expanding the frame to the deployed clot engagement configuration.

With reference to FIG. 36c and FIG. 36d the microcatheter 810 is withdrawn and unsheathes the debonding element 852 which expands towards its intermediate diameter. The expansion of the debonding element occurs adjacent the distal end of the occlusion 840. The orientation of the debonding element 852 is checked and if necessary the orientation of the debonding element adjusted. The debonder 851 is withdrawn into the distal body of the occlusion 840. The basket 801 is retracted and engaged with the clot 840. The debonder 851 is advanced slightly and the debonding element 852 engages with the clot, expands under the reaction force of the clot and in so doing sets up a shearing force on the body of the clot. The procedural steps comprise:
Deploying the debonding element 852 within the target vessel.
Orienting the debonding element 852.
Retracting the debonding element 852 into the body of the occlusion 840, wherein the retraction step comprises an incremental collapse of the debonding element 852.
Engaging the basket 801 with the distal end of the clot 840.
Engaging the debonding element 852 with the clot 840, said engagement comprising a spontaneous incremental expansion of the debonding element 852.

With reference to FIG. 36e the device 850 is shown with a distal portion 854 of the clot 840 sheared from the main body of the clot 840 and being forced into the mouth of the basket 801. The spontaneous expansion of the debonding element 852 when engaged with the clot helps the debonder 851 to shear away a portion 854 of the clot 840. This approach is particularly advantageous where the occlusion is an especially long occlusion. Occlusions of 30 mm are not unusual in cerebral vessels. Breaking the occlusion into chunks reduced the stress applied to the vessel wall and this reduces complications. It will be appreciated that the device 850 can be used to debond and capture short length occlusions without breaking up the clot. The steps associated with the shearing off and capture of the first chunk 854 of the occlusion 840 comprise:
Shearing off a segment 854 of the clot 840
Advancing the debonder with the sheared segment 854 distal while holding the basket 801 steadfast and forcing the sheared segment 854 into the basket 801.
Disengaging the debonding element 852 from the clot segment 854.

With reference to FIG. 36f and FIG. 36g the device is shown engaging with and capturing a second segment 855 of the clot 840. The debonder 851 is withdrawn proximally and the debonding element 852 is withdrawn into the remaining clot. When the user is satisfied with the size of the second clot segment 855 the debonder 851 is advanced slightly such that the debonding element 852 engages with the clot. The basket 801 is withdrawn proximally while holding the debonder 851 stationary and the basket 801 is engaged with the distal end of the clot segment 855. The debonder 851 is advanced while holding the basket 801 steadfast and the second clot segment 855 is sheared off and captured in the basket 801. The steps associated with capturing the second clot segment comprise:
Retracting the debonding element 852 into the body of the remaining occlusion 840, wherein the retraction step comprises an incremental collapse of the debonding element 852 as it engages with the occlusive material.
Engaging the basket 801 with the distal end of the second clot segment 855.
Engaging the debonding element 852 with the clot 840, in the body of the remaining clot, said engagement comprising a spontaneous incremental expansion of the debonding element 852.
Shearing off a second segment 855 of the clot 840.
Advancing the debonder with the sheared segment 855 distal while holding the basket 801 steadfast and forcing the sheared segment 855 into the basket 801.

In one embodiment the second segment comprises all of the remaining clot. In this case the method comprises the steps of:
Retracting the debonding element 852 through the remaining occlusion 840, wherein the retraction step comprises an incremental collapse of the debonding element 852 as it engages with the occlusive material, the debonding element 852 spontaneously expanding when the debonding element 852 emerges on the proximal side of the occlusion.
Engaging the basket 801 with the distal end of the remaining clot segment.
Engaging the debonding element 852 with the proximal face of the clot 840, said engagement comprising a spontaneous incremental expansion of the debonding element 852.
Shearing the remaining clot segment 855 from the wall of the vessel 841.
Advancing the debonder with the remaining clot segment 855 distal while holding the basket 801 steadfast and forcing the sheared segment 855 into the basket 801.

The remaining steps in the procedure are described in FIG. 36h and FIG. 36i. The debonder is withdrawn from the vessel segment. The basket is then withdrawn from the vessel segment and a final angiogram is completed. In one embodiment the steps comprise:
Retracting the debonding element 852 from the vessel segment.
Retracting the basket 801 with the captured clot from the vessel segment.
Removing the device 850 and the clot from the vasculature through the lumen of the guide catheter 811.

In another embodiment the device 850 and the microcatheter 810 are removed in concert. This approach allows the lumen of the microcatheter 810 to protect the vessel wall from the some of the frictional forces of the elongate tube 812 and the support member 805 during removal. The method comprises the steps of:
1. Retracting the debonder 851 until the expanded section of the debonding element 852 engages with the distal end of the microcatheter 810.
2. Locking the debonder 851 and the microcatheter 810 together.
3. Retracting the basket 801 until the frame 803 is adjacent the debonding element 852.
4. Retracting the microcatheter 810 and debonder 851 through another segment of vessel.
5. Repeat steps 3 and 4 until the debonder element is adjacent the tip of the guide catheter 811.
6. Retract the microcatheter 810, the debonder 851 and the basket through the lumen of the Guide catheter 811 and remove from the patient.
7. Conduct a final angiogram by flushing contrast media through the lumen of the guide catheter 811.

The debonder and the microcatheter can be easily locked together where a Touhy Borst fitting is connected to the proximal luer of the microcatheter.

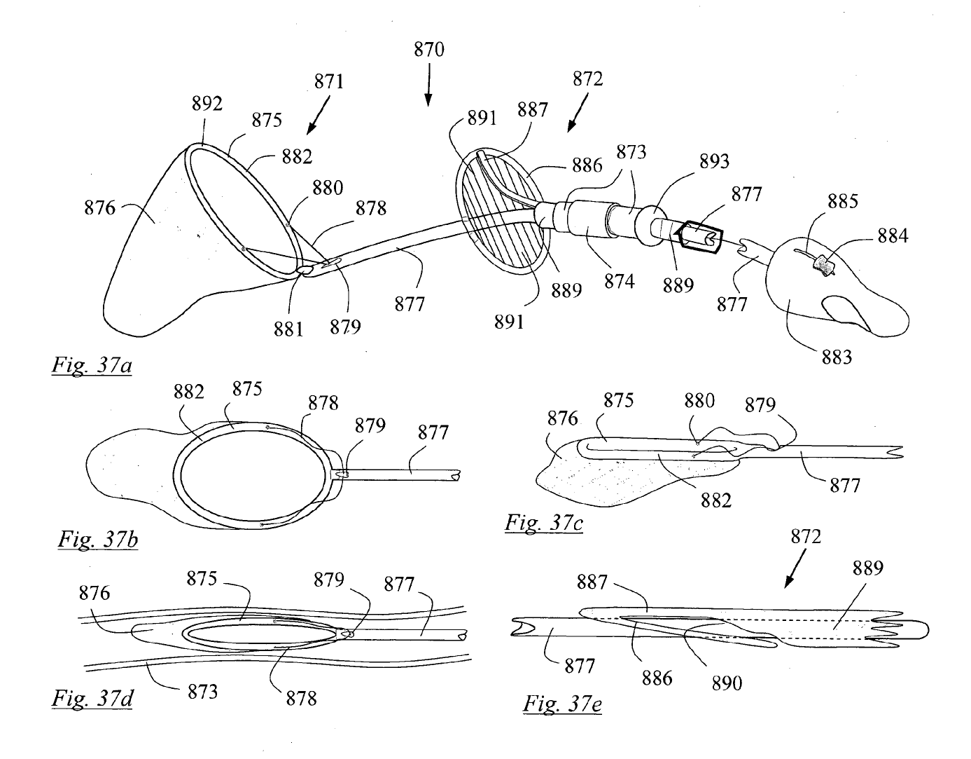

Another device 870 of the invention is described with reference to FIGS. 37a-e. FIG. 37a shows device 870 which comprises a basket assembly 871 and a debonding assembly 872. The basket assembly 871 comprises a frame 875, a net 876, a support member 877, pull cable 878, and a handle assembly 883. The support member 877 comprises a tubular member and the support member is connected to the frame at junction 881. The support member 877 comprises an inner lumen and an exit port 879. The pull cable extends from attachment point(s) 880 through the exit port 879 through the lumen of the support member 877 to the handle 883. In one embodiment the cable is interfaced with a slider 884 such that activation of the slider 884 causes the cable 878 to undergo tension and deactivation of the slider 884 reduces or removes tension from the cable 878. Tensioning the pull cables 878 causes the frame 875 to articulate about a region adjacent the junction 881. FIG. 37b and FIG. 37c show a top and side view respectively of the basket 871 with pull cable 878 in the untensioned state. FIG. 37d shows a top view of the basket 871 constrained in a vessel with pull cable 878 in the untensioned state. FIG. 37e shows a close-up of the debonding assembly 872 in the collapsed state for delivery.

In one embodiment the region of articulation is distal of the junction 881. In another embodiment the region of articulation is proximal of the junction 881. In another embodiment the region of articulation includes the junction. In one embodiment at least a portion of the region of articulation comprises a reduced section. In one embodiment the reduced section comprises a reduction in the width of the section. In another embodiment the reduced section comprises a reduction in the thickness of the section. In another embodiment the reduced section comprises a reduction in the cross sectional area of the section. In another embodiment the reduced section comprises a reduction in the stiffness of the material of the section.

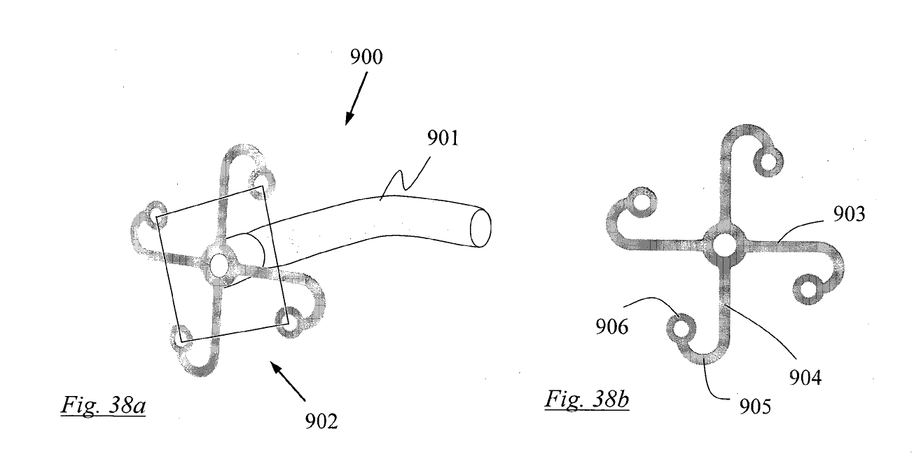

FIG. 38a shows another debonder 900 which may be used in conjunction with previous devices disclosed in the invention. The debonder is connected to an elongate tube 901 and has an engagement surface 902 for abutment with an occlusive clot. The engagement struts 903 in FIG. 38b have a radial section 904, a curved segment 905, and a termination section 906. This debonder configuration provides a large abutment surface area for an occlusive clot. It will be appreciated that tethers may be attached to the termination sections 906 of the engagement struts to provide additional engagement for the struts. FIG. 38b shows an end view of the same debonder 900 with tethers.

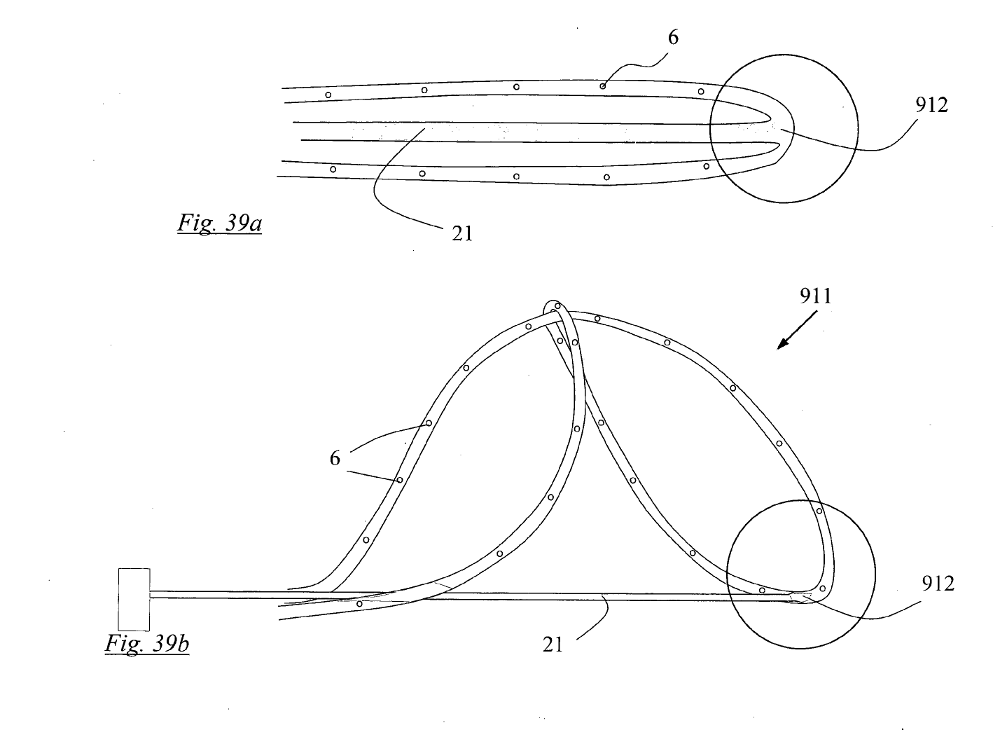

FIGS. 39a and 39b shows another basket frame 911 according to the invention. The basket frame has a tether connection 912 at the distal end. The frame may be constructed from a cut sheet of material. The frame may be constructed from cut tubular material. The frame may be constructed of wire material. The frame may be constructed of ribbon material. The material may be Nitinol. In the frame of FIGS. 39a and 39b the pull tether 21 in integrally attached to the frame. It will be appreciated that the pull tether may be attached by other means such as welding, laser welding, bonding, or tied to the basket frame. The frame in FIG. 39 has eyelets 6 as net attachment points.

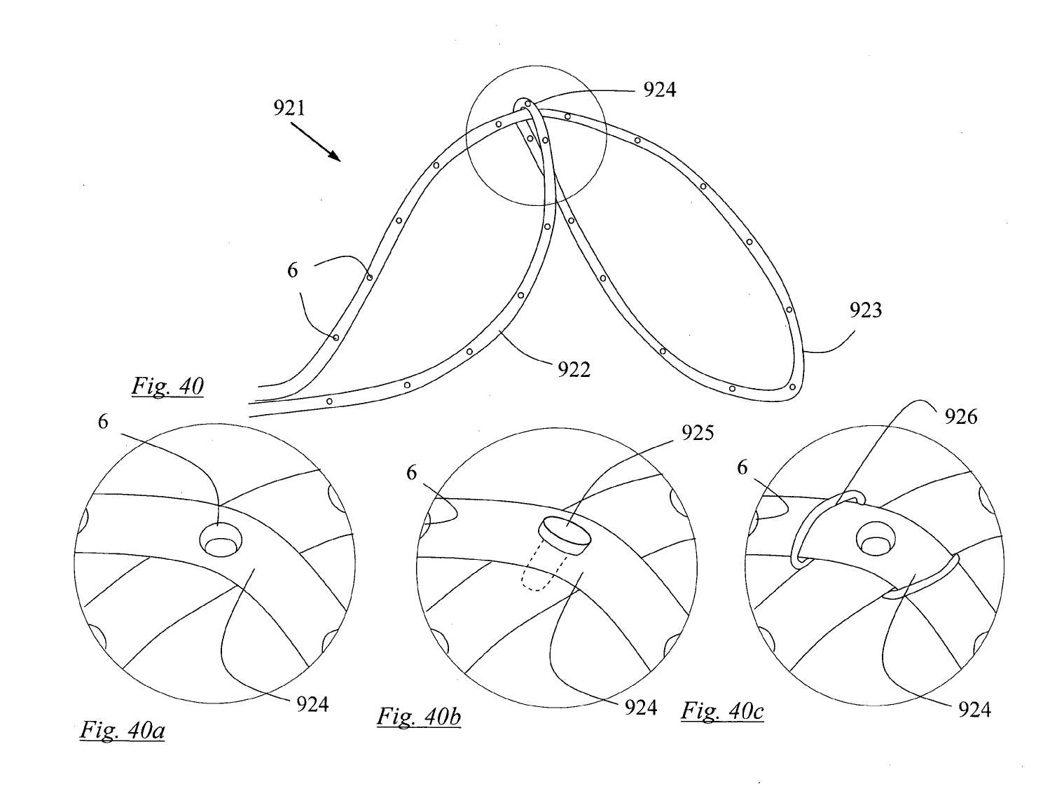

FIG. 40 shows another basket assembly 921 according to the invention. This basket frame has a proximal hoop 922 and a distal hoop 923. The frame may be constructed from a cut sheet of material. The frame may be constructed from cut tubular material. The frame may be constructed of wire material. The frame may be constructed of ribbon material. The material may be Nitinol. The struts of the proximal hoop and distal hoop are adjacent at a frame cross over 924. The struts of the proximal hoop and distal hoop may remain unconnected. FIG. 40a shows eyelets 6 of the proximal hoop struts and distal hoop struts may aligned at the frame cross over 924. FIG. 40b shows the frame cross over 924 wherein a pin 925 is inserted through eyelets 6. FIG. 40c shows the frame cross over 924 attached with a connecting wire 926. It will be appreciated that the means of connecting the struts shown in FIGS. 40b and 40c maintain the struts adjacent in the expanded configuration and in the collapsed configuration. The struts may move relative to each other in a scissors-like manner to move from an expanded configuration to a collapsed configuration.

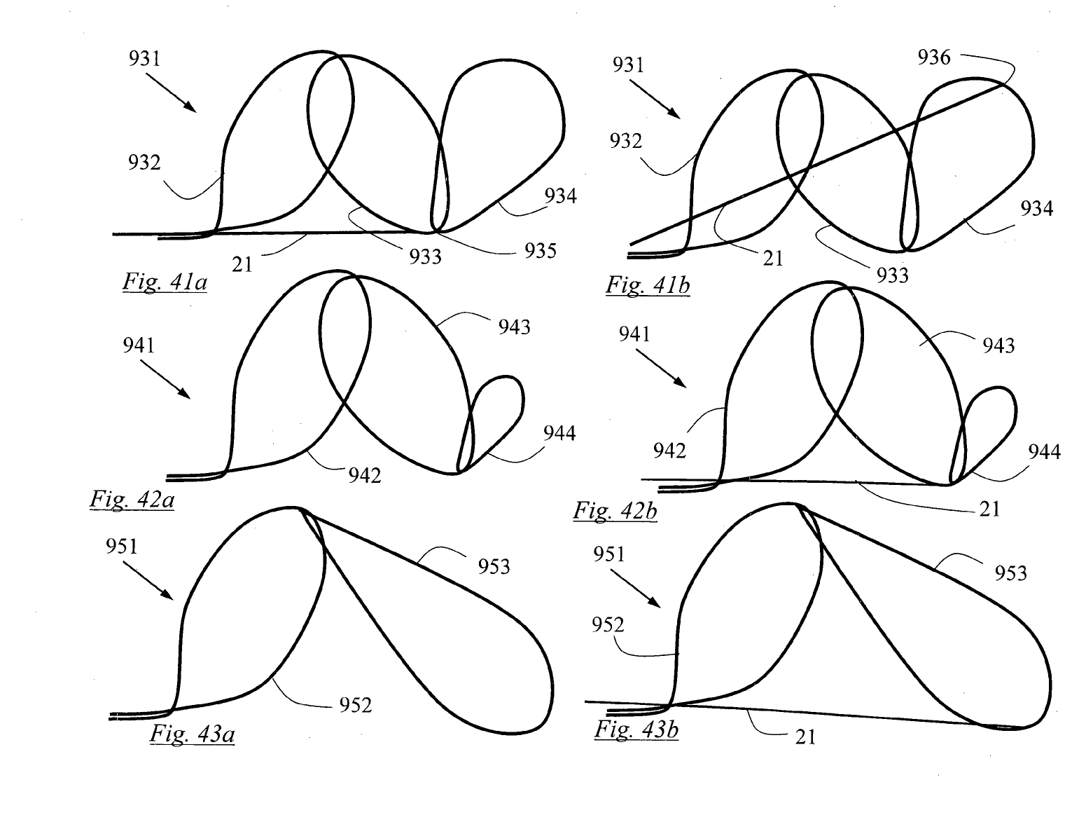

FIGS. 41a and 41b illustrate another basket assembly 931 of the invention in an expanded configuration. This basket assembly has a proximal loop 932, a middle loop 933, and a distal loop 934. In FIG. 41a a cable or pull tether 21 is connected at tether connection point 935 adjacent the middle loop 933 and distal loop 934 cross over point. In FIG. 42b the tether connection 936 is on the distal loop 934. The frame comprises three loops substantially the same circumference in FIG. 41. It will be appreciated that the plurality of loops are be incorporated in order to provide additional support to the basket assembly, and further loops can be incorporated.

FIGS. 42a and 41b illustrate another basket assembly 941 of the invention with a proximal loop 942, a middle loop 943, and a distal loop 944. In basket assembly 941 the distal loop 944 has a circumference smaller than that of the proximal or middle loops. FIG. 42a shows the frame without a tether and FIG. 42b shows a basket assembly 941 with a cable or pull tether 21. The bending stiffness of the smaller distal loop 944 gives the basket assembly rigidity in the axial direction to facilitate encapsulation of an occlusive clot.

FIGS. 43a and 43b show another basket assembly 951 of the invention with a proximal loop 952 and a distal loop 953. FIG. 43b has a cable or pull tether 21 connected to the distal loop for actuation. The distal loop 953 has a larger circumference than proximal loop 952, but in end view each loop will have substantially circular shapes to appose a vessel wall.

In FIGS. 41-43 it will be appreciated that basket assemblies may comprise cut sheet material, cut tube material, ribbon material or wire material.

Modifications and additions can be made to the embodiments of the invention described herein without departing from the scope of the invention. For example, while the embodiments described herein refer to particular features, the invention includes embodiments having different combinations of features. The invention also includes embodiments that do not include all of the specific features described.

The invention is not limited to the embodiments hereinbefore described, with reference to the accompanying drawings, which may be varied in construction and detail.

1. A device for the removal of clot obstructing the flow of blood through an arterial vessel, the device comprising an elongate member, a clot engaging element and a capture basket;
the elongate member extending in use from a point adjacent the target treatment site interior of the patient to a point exterior of the patient;
the capture basket comprising a frame and a net, and having an expanded and a collapsed configuration;
the clot engaging element comprising a plurality of struts having an expanded and a collapsed configuration, the plurality of struts forming a first section and a second section, said first section tapering outward and distally from the elongate member and connected to the second section, said second section comprising a plurality of cells defined by a plurality of struts and arranged around at least a portion of the circumference of an axis substantially parallel to that of the elongate member;
the clot engaging element and the capture basket being restrained in the collapsed configuration for delivery to the target site; and
the clot engaging element being located adjacent the distal end of the elongate member and proximal of the capture basket.

2. The device of claim 1 wherein the capture basket frame is self expanding.

3. The device of claim 1 wherein the clot engaging element is self expanding.

4. The device of claim 1 wherein the elongate member comprises a proximal section adjacent its proximal end and a distal section adjacent its distal end, said proximal section having a flexural stiffness greater than four times that of said distal section.

5. The device of claim 1 wherein the clot engaging element comprises a central axis and a contact surface, said central axis being substantially parallel to the elongate member, said contact surface being engagable with a clot and extending around at least a portion of the central axis.

6. The device of claim 5 wherein the contact surface extends around the entire circumference of the central axis.

7. The device of claim 1 wherein the plurality of cells of the second section of the clot engaging element are arranged around the entire circumference of an axis substantially parallel to that of the elongate member.

8. The device of claim 1 wherein the elongate member comprises an outer tubular element and an inner operating element.

9. The device of claim 8 wherein the inner operating element is movable relative to the outer tubular element and extends both proximally and distally of the outer tubular element.

10. The device of claim 9 wherein the clot engaging element is attached to the distal section of the outer tubular element and the capture basket is attached to the distal section of the inner operating element.

11. The device of claim 1 wherein the capture net frame is expandable to conform to the inner diameter of the vessel in which it is deployed.

12. The device of claim 11 wherein the elongate member contains an operating cable which is connected to an element of the capture net frame and which can be advanced or retracted relative to the elongate member to control the degree of expansion of the frame.

13. The device of claim 1 wherein the clot engaging element is expandable to conform to the inner diameter of the vessel in which it is deployed.

14. The device of claim 1 wherein the net comprises a braided, knitted or filament wound net and the net has an open end and a closed end.

15. The device of claim 1 wherein the clot engaging element comprises one or more tether segments which extend between some or all of the plurality of struts.

16. The device of claim 1 wherein the clot engaging element is laser cut from a tube or sheet.
